(19) United States
(12) Patent Application Publication
(10) Pub. No.: US 2005/0282208 A1
(43) Pub. Date: Dec. 22, 2005

(54) Cellular phenotype

(75) Inventors: Cynthia Lynn Adams, San Carlos, CA (US); Reginald Norman de la Rosa, Concord, CA (US); Shyamlal Ramchandani, San Francisco, CA (US)

Correspondence Address: BEYER WEAVER & THOMAS LLP, P.O. BOX 70250, OAKLAND, CA, 94612-0250, US

(73) Assignee: Cytokinetics, Inc.

(21) Appl. No.: 11/155,934

(22) Filed: Jun. 16, 2005

Related U.S. Application Data
(60) Provisional application No. 60/580,749, filed on Jun. 18, 2004.

(51) Int. Cl. C12Q 1/68; G06F 19/00

(52) U.S. Cl. 435/6; 435/455; 435/325; 702/20

(57) ABSTRACT

Phenotypes and the cells that exhibit those phenotypes are described. The phenotype may be established as a “snapshot” of the cells at a particular time or it may be established as a variation in features over time, or as some combination of these “static” and “dynamic” characterizations. The phenotype may be characterized by at least the following features: (a) chromosomes that approach metaphase but fail to separate and maintain alignment compared to a control cell or cell population; (b) a bipolar spindle that is at least about 10% longer than a corresponding metaphase mitotic spindle from the control cell or cell population; and (c) during interphase the cell or population of cells exhibits a phenotype that is substantially similar to that of the interphase cells of the control cell or cell population.

### CROSS-REFERENCE TO RELATED APPLICATIONS

This application claims benefit of U.S. Provisional Patent Application No. 60/580,749, filed Jun. 18, 2004, naming Adams et al. as inventors and titled “Cellular Phenotype,” which is incorporated herein by reference for all purposes.

### BACKGROUND

This invention relates to particular cellular phenotypes and to the cells and populations of cells that exhibit such phenotypes. The invention also relates to methods, apparatus, and computer program products that identify and/or make use of the phenotypes.

It is often desirable to characterize a cell or cell population by its phenotype. A cell's phenotype may change when exposed to a new stimulus or a change in the level of exposure to such stimulus. A given cell line may exhibit one phenotype when exposed to a particular compound and a different phenotype when exposed to a related compound. Temperature, culture conditions, exposure time, concentration and a number of other parameters can also influence the phenotype of a cell line. In addition, a compound may exhibit a different phenotype in a different cell line.

Certain phenotypes are manifestations of a stimulus' mechanism of action. As such they can help identify the mechanism of action of a stimulus under investigation such as a drug candidate. Hence, studies of phenotypic variation are valuable in drug discovery research. Specifically, a drug candidate may be characterized by its ability to elicit a particular phenotype, which indicates activity against a particular cellular target. In addition, certain phenotypic variations may indicate that a candidate has a potential side effect. When a candidate elicits a phenotypic change unrelated to the relevant target, it may be an indication that the candidate has a side effect. For additional discussion of how phenotypes are used in drug discovery, see U.S. patent application Ser. No. 10/621,821, filed Jul. 16, 2003, by Kutsyy et al., and titled “METHODS AND APPARATUS FOR INVESTIGATING SIDE EFFECTS,” which is incorporated herein by reference for all purposes.

The potential of phenotypic studies has not been realized. Some phenotypes associated with particular mechanisms of action, side effects, etc. have yet to be characterized or even observed. New avenues of cell biology research are yielding novel phenotypes having utility in drug discovery and other areas.

### SUMMARY

Generally, this invention relates to specific phenotypes and the cells that exhibit these phenotypes. Note that the concept of a “phenotype” includes characterizations of morphological features (size, shape, distribution/concentration of cell components, etc.), as well as the gross features of a cell population (motility, arrest in a particular stage of the cell cycle, growth and division rate, death rate, etc.). The phenotype may be established as a “snapshot” of the cells at a particular time or it may be established as a variation in features over time, or as some combination of these “static” and “dynamic” characterizations. It may also be defined in terms of changes that occur in response to various levels or doses of a particular stimulus. In such cases, the phenotype is represented, at least in part, as a stimulus-response path. Further, the phenotype may be defined over multiple cell lines, with some lines showing a greater susceptibility to particular phenotypic features than other cell lines.

One aspect of the invention provides a “rice” phenotype embodied in cell or a population of cells. The term “rice” describes certain characteristics of the phenotype and is not limited to any particular type of cell line. The rice phenotype of this invention may be characterized by at least the following features: (a) chromosomes that approach metaphase but fail to separate and maintain alignment compared to a control cell or cell population; (b) a bipolar mitotic spindle (produced as the chromosomes approach metaphase) that is at least about 10% longer than a corresponding metaphase mitotic spindle from the control cell or cell population; and (c) during interphase, the cell or population of cells exhibits a phenotype that is substantially similar to that of the control cell or cell population. Examples of other features that may be used to characterize the “rice” phenotype include following: (i) a bent mitotic spindle in some cells of the population, (ii) a higher percentage of the cells in the cell population that die prematurely in comparison to the control cell or cell population, (iii) cells that die by apoptosis upon reaching a mitotic state, and (iv) cells that die by apoptosis after their DNA decondenses.

In addition, stimuli that produce the rice phenotype do so selectively in some cell lines and not in others, or at least do so to a significantly lesser degree in the others. For example, A549 cells are less susceptible to stimuli that produce the rice phenotype than are DU145 and SKOV3 cells.

Another aspect of the invention pertains to particular eukaryotic cells (e.g., mammalian cells) or cell populations that exhibit the rice phenotype. These cells or populations will possess at least the features identified above. Typically, the rice phenotype will be produced by applying a stimulus to the cell or cell population that does not initially exhibit the rice phenotype. The stimulus induces a transformation to produce the rice phenotype. In some embodiments, applying the stimulus comprises administering a compound to the cells or population(s).

The invention also pertains to methods and apparatus used in to investigate, characterize, or otherwise quantify, an effect under investigation for its ability to produce a rice phenotype of this invention. One method aspect of the invention produces a transformation in the phenotype of a cell or cell population by (a) exposing the cell or cell population to a stimulus; and (b) allowing the stimulus to interact with the cell or cell population in a manner that transforms the cell or cell population to give rise a phenotype having at least some of the features described above. The method may further involve (c) imaging the cell or cell population to capture features that characterize the phenotype of the cell or cell population; and (d) analyzing the image to determine whether the cell or cell population exhibits the phenotypic features specified in (b), to thereby determine whether the compound produces the transformation. In many cases, the stimulus involves exposure to a particular compound or group of compounds.

Apparatus of the invention may include devices for providing cells (e.g., cell cultures in multi-well plates), delivering stimulus to the cells (possibly in carefully metered amounts), imaging the cells before, during, and/or after exposure to the stimulus, analyzing the image, or any combination of such devices.

Another aspect of the invention provides a method of characterizing a cell or a cell population based on phenotype. The method may be characterized by the following sequence: (a) receiving data characterizing the phenotype of the cell or cell population; (b) analyzing the data to determine whether the cell or cell population possesses some or all of the phenotypic features identified above; and (c) characterizing the cell or cell population as having a rice phenotype when the cell or cell population is found to possess at least a requisite set of the features specified above. Note that when phenotypic data is collected across multiple cell lines, the information can be used to characterize the specificity of a treatment.

Another aspect of the invention pertains to computer program products including machine-readable media on which are stored program instructions for implementing at least some portion of the methods described above. Any of the methods of this invention may be represented, in whole or in part, as program instructions that can be provided on such computer readable media. In addition, the invention pertains to various combinations of data and associated data structures generated and/or used as described herein.

These and other features and advantages of the present invention will be described in more detail below with reference to the associated figures.

### BRIEF DESCRIPTION OF THE DRAWINGS

FIG. 1A is a table showing that the mitotic spindle in some phenotypes of this invention is elongated in comparison to metaphase spindles of control phenotypes as taken by manual measurements.

FIG. 1B is a table showing spindle length as automatically measured in images of SKOV3 cells treated with various compounds that showed an mitotic index of >20%. Rice data was averaged from 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester at 40, 22, 13, and 7 uM, and 4-(3-Bromo-4-hydroxy-5-methoxy-phenyl)-1,6-dimethyl-2-oxo-1,2,3,4-tetrahydro-pyrimidine-5-carboxylic acid cyclopentyl ester at 40 and 22 uM.

FIG. 2A presents tubulin marker images of control cells (top image) and test cells treated with 4-(3-Bromo-4-hydroxy-5-methoxy-phenyl)-1,6-dimethyl-2-oxo-1,2,3,4-tetrahydro-pyrimidine-5-carboxylic acid cyclopentyl ester at 40 μM for 24 hours prior to imaging (bottom image).

FIG. 2B shows how mitotic spindle length can be measured from pole to pole.

FIG. 3 is a schematic depiction of the mathematical approach to characterizing mitotic spindles as bent using circular variance.

FIG. 4 shows a time-lapse montage of GFP-histone 2B in SKOV3 cells every 10 minutes. The montages are provided for control phenotypes (right panel) and rice phenotypes in the presence of 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester at 33 uM (left panel).

FIG. 5A is a graph showing how mitotic index (which measures a compound's ability to cause mitotic arrest) varies as a function of a phenotypic “distance” from a normal interphase phenotype for phenotypes of this invention and certain other phenotypes. The data is from represenative compounds capable of inducing the Rice phenotype at multiple concentrations (40 uM maximum concentration), Taxol (0.8 uM maximum concentration), and control.

FIG. 5B is a profile showing how the rice phenotype is more similar to control for various interphase phenotypic features, while Taxol features vary to a greater degree when the mitotic index for all compounds is 10%.

FIG. 6A shows DNA in interphase cells exhibiting a normal phenotype and the phenotype of this invention.

FIG. 6B shows tubulin and DNA images of interphase cells exhibiting a normal phenotype and the rice phenotype of this invention.

FIG. 7A is a pair of graphs showing that a compound producing the rice phenotype has a limited effect on pertinent features in A549 cells, while having significant effects on mitotic index in the DU145 and SK0V3 cell lines.

FIG. 7B shows two images of DU145 cells, one exhibiting the rice phenotype and the other not. The compounds used induce the shown phenotypes both appeared (at least superficially) to produce a rice-like phenotype in SK0V3 cells. But the one compound shown in the lower panel did not produce a similar phenotype in the DU145 cells, and hence clearly did not produce the cell line specificity associated with the rice phenotype.

FIG. 8A shows a time-lapse montage of a SKOV3 cell exhibiting the rice phenotype undergoing DNA decondensation and then apoptosis.

FIG. 8B is a bar chart showing (phenotypes for rice and for inhibitors of two mitotic kinesins) the relative numbers of mitotically arrested cells undergoing apoptosis directly from mitosis and undergoing decondensation first.

FIG. 9 is a flow chart illustrating an embodiment of a general method employed to quantitatively determine whether a stimulus gives rise to the rice phenotype.

FIG. 10 is a flow chart illustrating cell sample preparation activities of the method illustrated by FIG. 9 in greater detail.

FIG. 11 is a flow chart illustrating image capture and processing activities of the method illustrated in FIG. 9 in greater detail.

FIG. 12 is a schematic block diagram of an embodiment of an image capture and image processing system suitable for carrying out some of the activities illustrated in FIG. 11.

FIG. 13 is a simplified block diagram of a computer system that may be used to implement various aspects of this invention, including characterizing cellular phenotypes, determining whether a given phenotype is a rice phenotype, and calculating distances between control and test phenotypes using “signatures” of those phenotypes.

### DESCRIPTION OF A PREFERRED EMBODIMENT

I. Introduction

As indicated, this invention pertains to phenotypes that were not previously observed. They may arise from a unique type of disruption to the mitotic apparatus in eukaryotic cells, although the invention is not limited to phenotypes arising from any particular stimulus. Because the phenotypes have mitotic spindles that are frequently elongated and curved giving the appearance of rice grains, the phenotypes of this invention are referred to herein as “rice phenotypes.” The following features are generally characteristic of a rice phenotype: an elongated mitotic spindle, a substantially unperturbed interphase phenotype, and chromosomes that fail to congress normally or reach an anaphase state (i.e., the chromosomes fail to separate and move toward the poles of the spindle). Typically, though not necessarily, all of these features are present in a phenotype of this invention. Additionally, the some of the cells exhibiting the rice phenotype commonly have a bent mitotic spindle. Another interesting observation is that the phenotype is pronounced only in certain cell lines. That is, treatments that produce the phenotype in some cell lines do not produce it other cell lines, or do so only at significantly higher levels of exposure (e.g., higher concentrations of a drug). Among the cell lines that do not readily exhibit the rice phenotype are the following: A549 cells (a human lung carcinoma cell line), and HeLa cells (a human cervical carcinoma cell line). This observed cell line selectivity is not seen in other phenotypes resulting from interference with mitosis with compounds such as Taxol. Another common feature of cells exhibiting the rice phenotype is death following mitotic arrest, usually by apoptosis.

Note that characteristics of the rice phenotype are defined with respect to a control cell or population of cells, which has not been exposed to a stimulus that produces the novel phenotype. Aside from exposure to such stimulus, the control and the test cells should be similar in terms of genotype and history (source, culturing, environment influences, etc.).

Any given cell that exhibits the features identified above may be characterized as having a rice phenotype of this invention. However, a population of cells may also be said to possess the rice phenotype if some number or a percentage of its member cells exhibit the above features (when compared to a control population that have not been exposed to a stimulus that produces the rice phenotype). For example, the phenotype may be present if on average the members of the population exhibit the features. Further, it has been observed that certain interesting phenotypic characteristics typically occur only in a fraction of a cell population exhibiting the rice phenotype. One example is a bent mitotic spindle.

As explained below, phenotypes of this invention may be identified by eye, manual measurement, automated measurement and analysis, etc. However, certain specific aspects of this invention pertain to automated image analysis techniques that identify phenotypes of this invention. Such techniques may make use of markers for cellular components that assume interesting structures during mitosis and interphase states. Examples of such components include histones, DNA, tubulin, and certain other cytoskeletal components such as actin.

The rice phenotype may be generated by any of a number of different stimuli. It has been found that exposure to a particular class of compounds generates the heretofore unknown phenotype. These compounds include, for example, those described in U.S. Patent Application No. 60/512,494 filed Oct. 16, 2003, which is incorporated herein by reference for all purposes.

II. Definitions

Some of the terms used herein are not commonly used in the art. Other terms may have multiple connotations in the art. Therefore, the following definitions are provided as an aid to understanding the description herein. The invention as set forth in the claims should not necessarily be limited by these definitions.

The term “component” or “component of a cell” refers to a part of a cell having some interesting property that can be characterized by image analysis to derive biologically relevant information. General examples of cell components include biomolecules and subcellular organelles. Specific examples of biomolecules that can serve as cell components include specific proteins and peptides, lipids, polysaccharides, nucleic acids, etc. Sometimes, the relevant component will include a group of structurally or functionally related biomolecules. Alternatively, the component may represent a portion of a biomolecule such as a polysaccharide group on a protein, or a particular subsequence of a nucleic acid or protein. Collections of molecules such as micells can also serve as cellular components for use with this invention. And subcellular structures such as vesicles and organelles may also serve the purpose.

The term “marker” or “labeling agent” refers to materials that specifically bind to and label cell components. These markers or labeling agents should be detectable in an image of the relevant cells. Typically, a labeling agent emits a signal whose intensity is related to the concentration of the cell component to which the agent binds. Preferably, the signal intensity is directly proportional to the concentration of the underlying cell component. The location of the signal source (i.e., the position of the marker) should be detectable in an image of the relevant cells.

Preferably, the chosen marker binds specifically with its corresponding cellular component, regardless of location within the cell. Although in other embodiments, the chosen marker may bind to specific subsets of the component of interest (e.g., it binds only to sequences of DNA or regions of a chromosome). The marker should provide a strong contrast to other features in a given image. To this end, the marker may be luminescent, radioactive, fluorescent, etc. Various stains and compounds may serve this purpose. Examples of such compounds include fluorescently labeled antibodies to the cellular component of interest, fluorescent intercalators, and fluorescent lectins. The antibodies may be fluorescently labeled either directly or indirectly.

The term “stimulus” refers to something that may influence the biological condition of a cell. Often the term will be synonymous with “agent” or “manipulation” or “treatment.” Stimuli may be materials, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include non-ambient temperature, non-ambient pressure, acoustic energy, electromagnetic radiation of all frequencies, the lack of a particular material (e.g., the lack of oxygen as in ischemia), temporal factors, etc.

A particularly important class of stimuli in the context of this invention is chemical compounds, including compounds that are drugs or drug candidates and compounds that are present in the environment. The biological impact of chemical compounds is manifest as clear phenotypic changes such as those producing phenotypes of this invention. Related stimuli involve suppression of particular targets by siRNA or other tool for preventing or inhibiting expression.

The term “phenotype” generally refers to the total appearance and behavior of a cell or multi-cellular organism. The phenotype results from the interaction of an organism's genotype and the environment. Cellular phenotypes may be defined in terms of various qualitative and quantitative features. These features may be captured and stored in images and in numeric and/or symbolic representations in processing systems (e.g., computers) and data storage media (whether or not directly associated with a computer system). For certain embodiments of this invention, the phenotype is a characteristic of a population of similarly situated cells (having a common environment and/or history of interactions with the environment). Thus, the phenotype may be manifest by particular visible features and/or behaviors that vary depending upon the state of the cell. For example, a phenotype may be manifest by one feature while in the mitotic portion of the cell cycle and a different, even unrelated, feature while in interphase portion of the cell cycle.

Often a particular phenotype can be correlated or associated with a particular biological condition or mechanism of action resulting from exposure to a stimulus. Generally, cells undergoing a change in biological conditions will undergo a corresponding change in phenotype. Thus, cellular phenotypic data and characterizations may be exploited to deduce mechanisms of action and other aspects of cellular responses to various stimuli.

A selected collection of data and characterizations that represent a phenotype of a given cell or group of cells is sometimes referred to as a “quantitative cellular phenotype.” This combination is also sometimes referred to as a phenotypic fingerprint or just “fingerprint.” The multiple cellular attributes or features of the quantitative phenotype can be collectively stored and/or indexed, numerically or otherwise. The attributes are typically quantified in the context of specific cellular components or markers. Measured attributes useful for characterizing an associated phenotype include morphological descriptors (e.g., size, shape, and/or location of the organelle), cell count, motility, composition (e.g., concentration distribution of particular biomolecules within the organelle), and variations in the degree to which different cells exhibit particular features. Often, the attributes represent the collective value of a feature over some or all cells in an image (e.g., some or all cells in a specific well of a plate). The collective value may be an average over all cells, a mean value, a maximum value, a minimum value or some other statistical representation of the values.

The quantitative phenotypes may themselves serve as individual points on “response curves.” A phenotypic response to stimulus may be determined by exposing various cell lines to a stimulus of interest at various levels (e.g., doses of radiation or concentrations of a compound). In each level within this range, the phenotypic descriptors of interest are measured to generate quantitative phenotypes associated with levels of stimulus.

The term “path” or “response curve” refers to the characterization of a stimulus at various levels. For example, the path may characterize the effect of a chemical applied at various concentrations or the effect of electromagnetic radiation provided to cells at various levels of intensity or the effect of depriving a cell of various levels of a nutrient. Mathematically, the path is made up of multiple points, each at a different level of the stimulus. In accordance with this invention, each of these points (sometimes called signatures) is preferably a collection of parameters or characterizations describing some aspect of a cell or collection of cells. Typically, at least some of these parameters and/or characterizations are derived from images of the cells. In this regard, they represent quantitative phenotypes of the cells. In the sense that each point or signature in the path may contain more than one piece of information about a cell, the points may be viewed as arrays, vectors, matrices, etc. To the extent that the path connects points containing phenotypic information (separate quantitative phenotypes), the path itself may be viewed as a “concentration-independent phenotype.” The generation and use of stimulus response paths is described in more detail in U.S. patent application Ser. No. 09/789,595, filed Feb. 20, 2001 naming Vaisberg et al., and titled, “CHARACTERIZING BIOLOGICAL STIMULI BY RESPONSE CURVES,” and U.S. Patent Application No. 60/509,040, filed on Jul. 18, 2003, naming V. Kutsyy, D. Coleman, and E. Vaisberg as inventors, and titled, “Characterizing Biological Stimuli by Response Curves,” both of which are incorporated herein by reference for all purposes.

As used herein, the term “feature” refers to a phenotypic property of a cell or population of cells. As indicated, individual quantitative phenotypes (fingerprints) are each comprised of multiple features. The terms “descriptor” and “attribute” may be used synonymously with “feature.” Features derived from cell images include both the basic “features” extracted from a cell image and the “biological characterizations” (including biological classifications such as cell cycle states). The latter example of a feature is typically obtained from an algorithm that acts on a more basic feature. The basic features are typically morphological, concentration, and/or statistical values obtained by analyzing a cell image showing the positions and concentrations of one or more markers bound within the cells.

III. Phenotypic Characteristics

1. Elongated Spindle

In phenotypes of this invention, the mitotic spindle is frequently found to be longer than that of a control phenotype. In a typical case, the difference in length is at least about 10% and it is not uncommon for the difference to be about 20% or more.

More specifically, the rice phenotype spindle is typically longer than a metaphase spindle observed in control cells. Because most cells exhibiting the rice phenotype do not progress beyond metaphase, the spindle length comparison is typically made with respect to control cells in metaphase. Note that in normal cells, the spindle length naturally increases as the cells transition from metaphase to anaphase and then on to telophase. But cells exhibiting the phenotype of this invention typically fail to progress past metaphase. Nevertheless their spindles quickly grow significantly longer than that of their control counterparts in metaphase. Often the spindle length associated with the rice phenotype is comparable to the spindle length of a control cell in anaphase.

The elongated spindle feature is not found in phenotypes produced by many types of stimulus that interfere with the mitotic apparatus, including the kinetochore. Examples of compounds that interfere with mitosis but do not produce elongated metaphase spindles include Taxol and various compounds that interact with active sites on various kinetochore associated proteins or proteins involved in pre-metaphase arrest (e.g., KSP, CENP-E, RABK6, BubR1, and Aurora (AUR1, and AUR2)). Hence, phenotypes of this invention are surprisingly easy to distinguish from phenotypes produced by such compounds.

As is well known, the mitotic spindle originates in the cytoplasm during prophase. It includes fibers constructed of microtubules and microtubule-associated proteins. The fibers fall into two categories: polar fibers (the more numerous) extend from the poles of the spindle toward the equator and kinetochore fibers attached to the centromere of each chromosome and extend toward the spindle poles. More specifically, the kinetochore fibers attach to the kinetochore, which is a structure associated with the centromere. The kinetochore and its component proteins are involved in coordinating chromosome movement via microtuble assembly and disassembly. A typical spindle includes about 10⁸ tubulin molecules assembled into microtubules. Of course, other tubulin is present in a mitotic cell and there appears to be dynamic equilibrium between spindle microtubules and a pool of soluble tubulin molecules in mitotic cells.

In accordance with this invention, the length of a bipolar mitotic spindle may be measured as the separation distance between the spindle poles. To make such measurement, mitotic spindles are observed by a suitable technique such as one that employs a marker for a specific component of the spindle. For example, the technique may involve treating cells with a marker for tubulin and then imaging them under conditions that illuminate such marker. This facilitates automated identification of the mitotic spindle and measurement of its length using image analysis algorithms. Examples of tubulin markers include fluorescently labeled antibodies to tubulin (e.g., DM1-α, YL1-2, and 3A2 antibodies), cells expressing GFP (or the like) labeled tubulin, or microinjection of rhodamine labeled tubulin into live cells.

Of course, markers for other features of the mitotic spindle may be used in other embodiments. Distances between centromeres could also be measured using markers to gamma-tubulin, EB1, or antibodies to the centrosome for example. Further, it is possible in some embodiments to use a technique that does not rely on a specific marker. For example, in some cases, cells may be observed using simple light microscopy techniques such as differential-interference-contrast microscopy, phase-contrast microscopy, or polarized light microscopy or any other technique in which the mitotic spindle is easily distinguishable from other sub-cellular components.

Characterization of mitotic spindles in a population of cells is facilitated by first identifying the cells that are truly mitotic. This allows the procedure to consider tubulin and spindle length only in mitotic cells.

Mitotic cells may be identified by various techniques including techniques that identify condensed DNA. For example, cells can be classified as mitotic or interphase based on a combination of the size of nuclei and the amount of DNA in nuclei (as revealed by DNA staining using, for example, DAPI or Hoechst 33341 stains (available from Molecular Probes, Inc. of Eugene, Oreg.)). Mitotic cell DNA is generally smaller and brighter (i.e., captured images have higher mean and median pixel intensities) than DNA in interphase cells. Examples of some specific techniques identifying mitotic cells are described in U.S. patent application Ser. No. 09/729,754, filed Dec. 4, 2000, by Vaisberg et al., and titled “CLASSIFYING CELLS BASED ON INFORMATION CONTAINED IN CELL IMAGES,” which is incorporated herein by reference for all purposes.

In some cases, techniques that rely on the presence of condensed DNA might mischaracterize certain non-mitotic cells, such as some cells undergoing apoptosis, as mitotic. One method of specifically identifying mitotic cells, without mischaracterizing apoptotic cells, employs a marker for a phosphorylated histone, e.g., phospho-histone 3 (pH3). During mitosis, the histones in the nucleus become phosphorylated. Therefore, mitotic cells may be identified using a pH3 marker such as a fluorphore-labeled primary antibody to the phosphorylated histone.

Other methods of specifically identifying mitotic cells involve tubulin thresholding. In such methods, cells are marked with a tubulin marker. Only those cells exhibiting local tubulin marker intensity above a particular threshold (associated with microtubules in a spindle) are deemed to be mitotic. In non-mitotic cells, cellular tubulin is diffuse and does not form regions of locally high concentration, unlike the situation with mitotic cells, where microtubles form in the mitotic spindle.

In control cells metaphase spindles are distinguished by their characteristic property of being both bright and linear compared to all other DNA morphologies, while anaphase is identified by the appearance of two slightly less bright, but parallel linear sections of DNA (marking the separation of the duplicated DNA). These states are easily identified in fixed images, but are most obvious taken in context with multiple frames of a time-lapse movie.

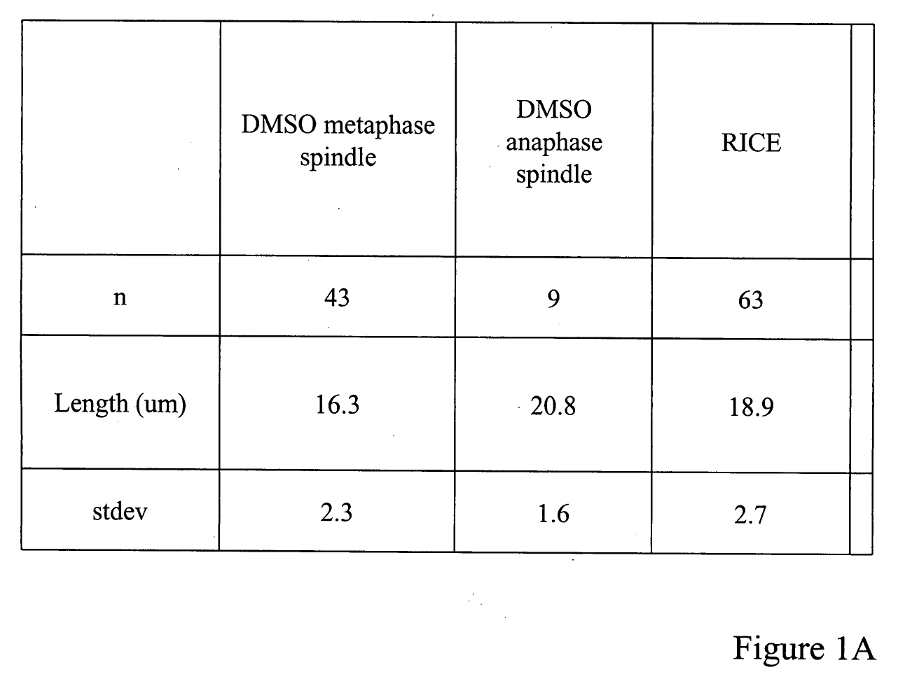

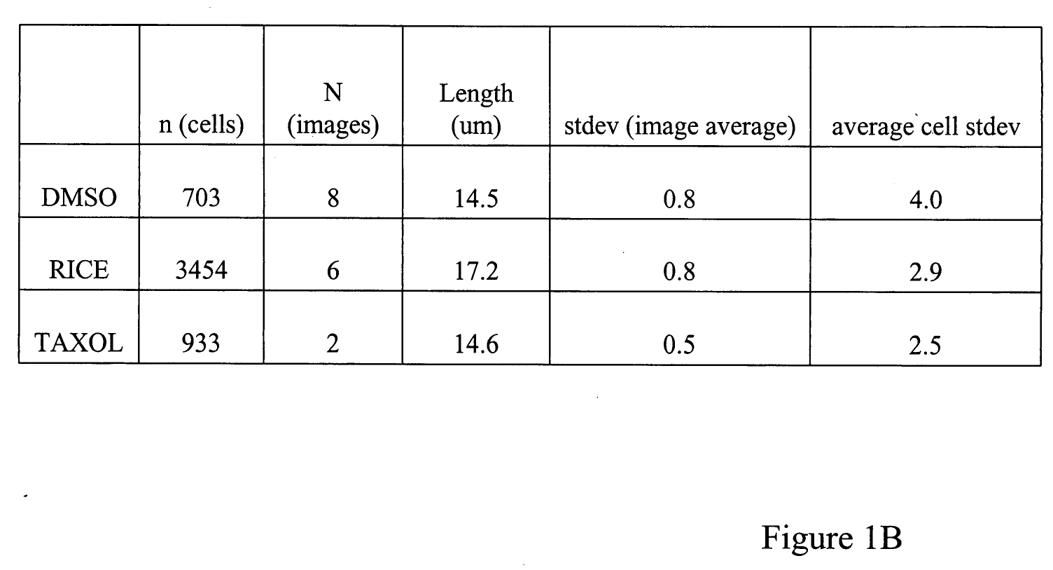

The table in FIG. 1A shows that the elongated spindle in some phenotypes of this invention is closer in length to an anaphase spindle (defined as when the daughter nuclei are distinct, but a cleavage furrow is still not marked by tubulin), which is about 20% longer than a spindle in metaphase. Note again that the DNA in cells exhibiting the rice phenotype typically fails to progress beyond metaphase. Hence, the daughter chromosomes usually fail to separate as in anaphase cells.

The data in this table was obtained for SKOV3 cells (ovarian cancer cell line) treated with DMSO (control) and a compound that produces the rice phenotype. Control phenotypes were produced by treating the cells with 0.4% DMSO for 24 hours. Rice phenotypes of this invention were produced by treating the cells with 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester at 40 uM for 24 hours. In the table, “n” is the number of cells that were analyzed to generate a mean length in micrometers as indicated and associated standard deviation. Spindle lengths were measured manually in this example.

Manual measurements were taken from one image each of DMSO, rice compound 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester at 40 uM, and a mitotic kinesin inhibitor at 40 uM using MetaMorph Imaging Software. Lines were drawn between what was manually determined to be the ends of the mitotic spindle. The software then calculated the distances for all lines drawn, and Microsoft Excel was used to calculate the averages.

Regarding the table of FIG. 1B, the software MetaMorph from Universal Imaging Corporation provided an automated measure of mitotic spindle length using fixed threshold and size filters on images of SKOV3 cells treated with compounds from the experiment described for FIG. 1A. Briefly, all compounds causing a mitotic index greater than 0.2 (i.e., the proportion of mitotic cells in the cell population is greater than 0.2) were analyzed. Automated threshold values were set to 1700-4095 (these values would change for images collected on a different day) and any object greater than 100 pixels was measured for multiple features, including long axis length using the Intgrated Morphometry Anaysis (IMA) tool. A log file was generated with the average and standard deviations of the lengths, and the averages for all of the images were calculated using Microsoft Excel.

Again 0.4% DMSO was used for control phenotypes. Test phenotypes were produced by exposing the cells to multiple different concentrations and compounds for 24 hours (see figure legend for conditions). In the table, N is the number of images considered, n is the total number of cells having measured mitotic spindles (across all images), “Length” is the mean mitotic spindle length across all cells in all images, “stdev (image average)” is the average of the standard deviations for each image, and “average cell stdev” is standard deviation across all cells considered.

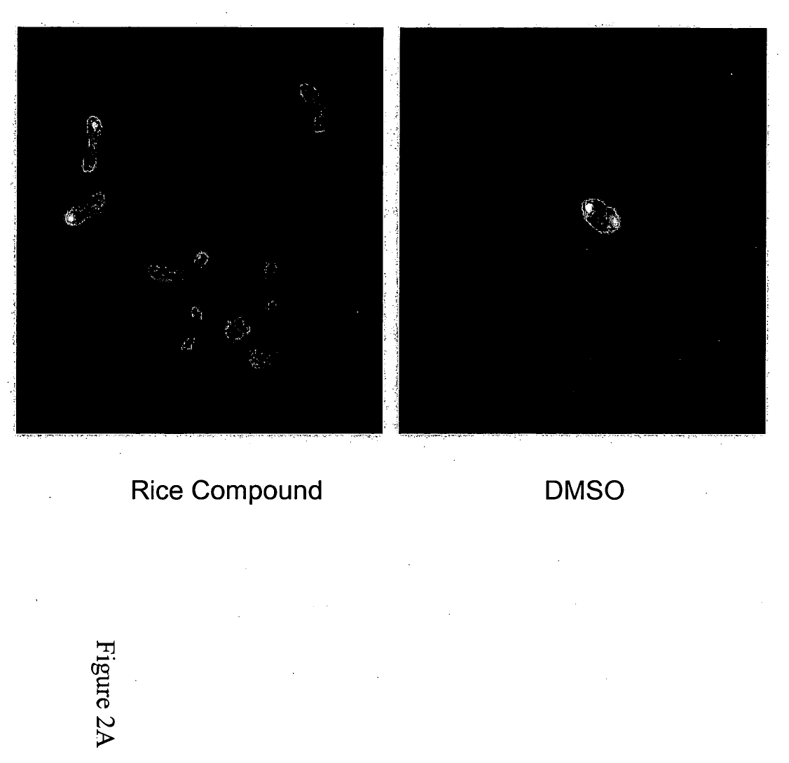

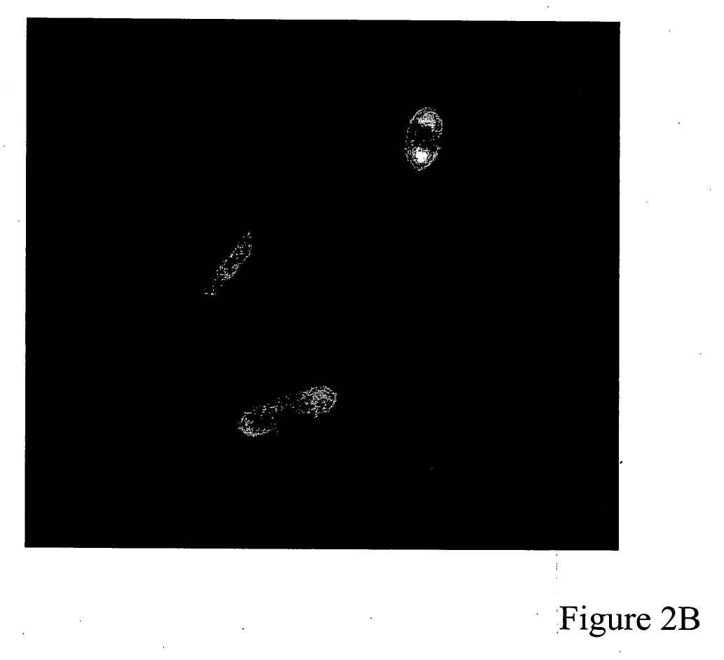

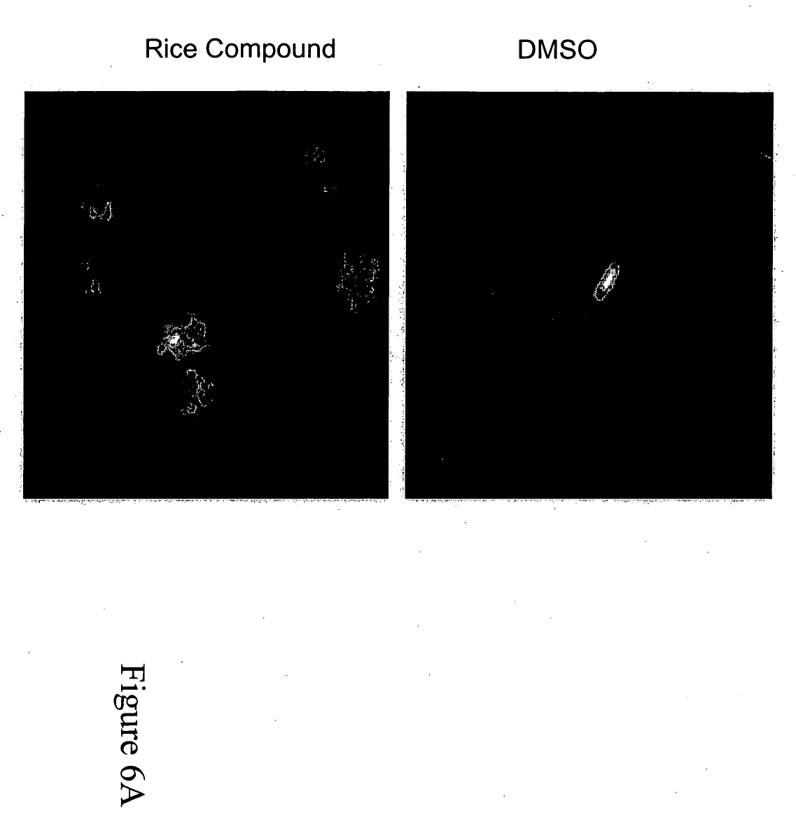

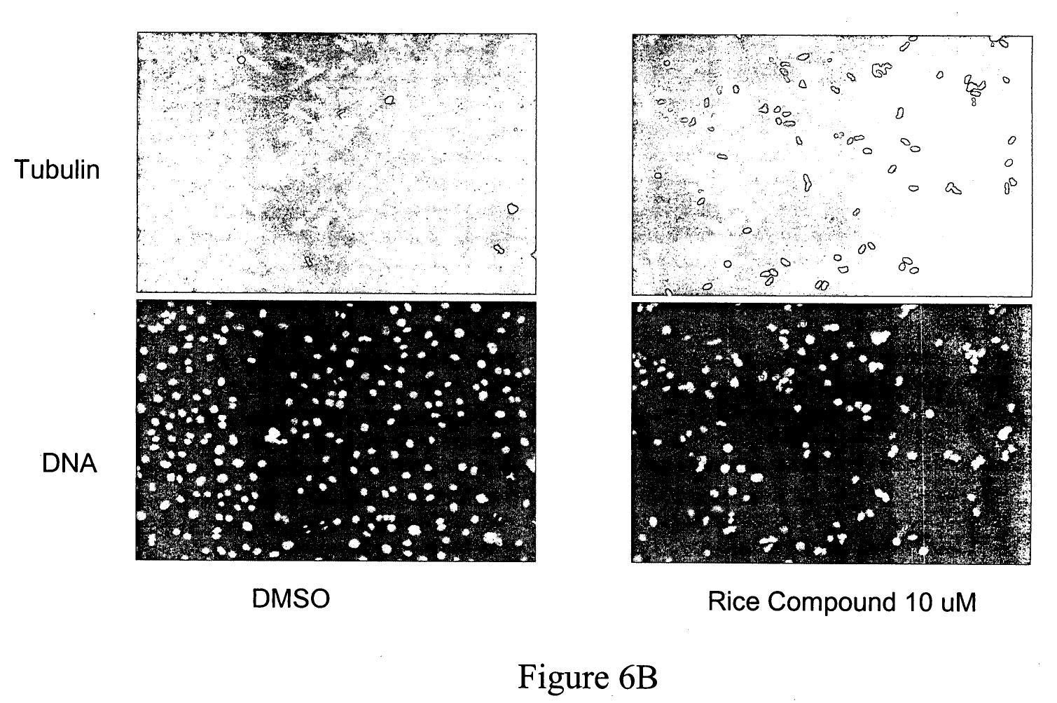

FIG. 2A presents images of control cells treated with 0.4% DMSO (upper image) and test cells treated with 4-(3-Bromo-4-hydroxy-5-methoxy-phenyl)-1,6-dimethyl-2-oxo-1,2,3,4-tetrahydro-pyrimidine-5-carboxylic acid cyclopentyl ester at 40 uM for 24 hours prior to imaging (lower image). All cells were treated with fluorophore labeled DM1α to show tubulin. The bright spots are locations of mitotic spindles. As shown, mitotic spindles in test cells are significantly longer than spindles in normal cells. Related FIG. 6A, described below, shows the DNA in the same cells.

FIG. 2B shows how spindle length can be measured from pole to pole. It can be appreciated that this spindle length can be easily obtained by manual or automated measurements. An example of an automated measurement technique works as follows. The cell image is initially segmented to identify individual cells in the image. Segmentation can be performed by various techniques including those that rely on identification of discrete nuclei and those that rely cytoskeletal proteins. Exemplary segmentation methods are described in U.S. Patent Publication No. US-2002-0141631-A1 of Vaisberg et al., published Oct. 3, 2002, and titled “IMAGE ANALYSIS OF THE GOLGI COMPLEX” and U.S. Patent Publication No. US-2002-0154798-A1 of Cong et al. published Oct. 24, 2002 and titled “EXTRACTING SHAPE INFORMATION CONTAINED IN CELL IMAGES,” both of which are incorporated herein by reference for all purposes. In a specific embodiment, an algorithm operates on tubulin marker signal in the segmented cells and uses the length of the long axis of a fitted ellipse for mitotic, or pH3 positive cells, to measure the spindle length.

2. Bent Mitotic Spindles

A significant proportion of the cells exhibiting the rice phenotype have bent mitotic spindles. Generally the same techniques described above to observe mitotic spindle length (e.g., imaging cells treated with a tubulin marker) may be employed to observe spindles for the purpose of determining whether they are bent.

In many cases, bent spindles are readily detected by either visual inspection or automated image analysis. Various metrics can be employed to distinguish bent from “straight” spindles. These include measures of curvature such as certain shape descriptors, and the like.

As the name suggests, circular variance represents the deviation of a particular shape or edge from a true circle. The goal is to distinguish generally straight or elongated shapes from generally circular shapes. Shapes with a greater degree of elongation will have a larger value of circular variance.

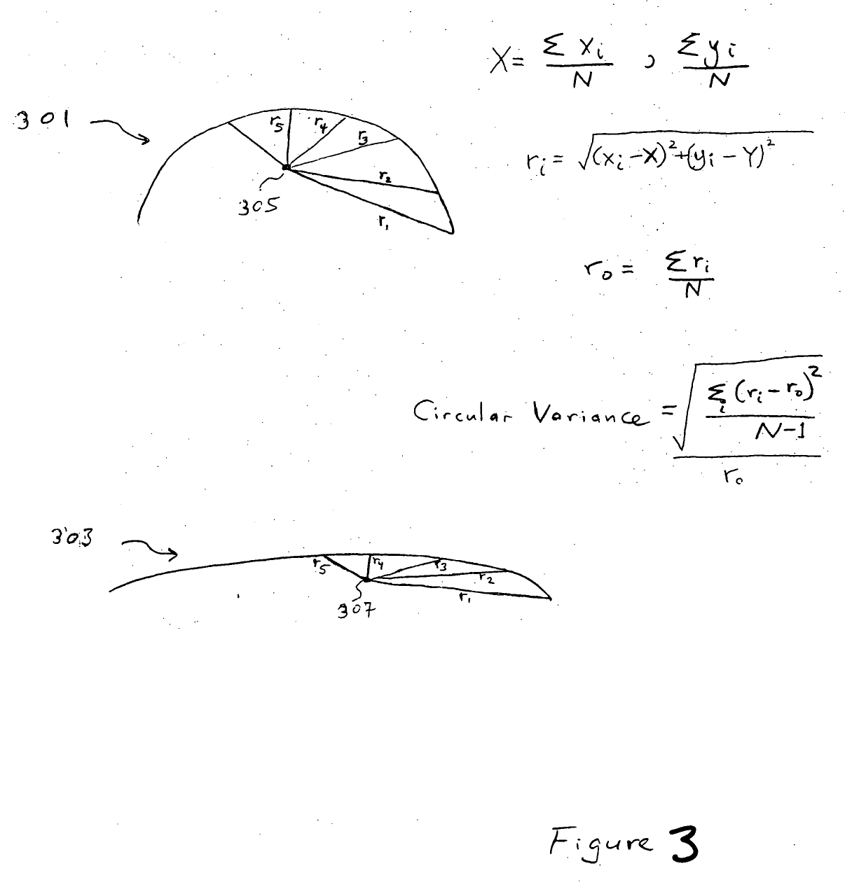

The concept of circular variance is illustrated in FIG. 3. Initially, the method calculates a centroid for a centerline path of the mitotic spindle under consideration in the image. The centroid (X, Y) represents the coordinate of the mean value of X and the mean value of Y in the spindle shape. In a relatively bent spindle 301 depicted in FIG. 3, the centroid is given by a point 305. In a relatively elongated spindle 303, the centroid is represented by a point 307.

Once the centroid of a spindle is identified, the radii between the centroid and each point on the spindle path are calculated. As shown in FIG. 3, these are indicated by the rᵢ (r₁, r₂, r₃ . . . ). From these radii, a mean radius value r₀ is calculated for the spindle under consideration. With this mean value and the individual radii, the circular variance can be calculated from the expression shown in FIG. 3. Note that the parameter “N” represents the total number of points considered in the spindle path. Spindles with a relative small range in the value of their individual radii will give smaller values of circular variance and thereby be characterized as bent in accordance with this invention. In some cases, “bent” mitotic spindles are identified by a circular variance of at most about 0.5. Of course, this is only an example and for certain cell lines and treatments other variance values will be appropriate. Note that circular variance is only one of many shape descriptors that can be used to characterize the shape of spindles in accordance with this invention.

As an example of cells having bent mitotic spindles, see the two central mitotic cells shown in lower panel of FIG. 2A. As shown there, some mitotic spindles (as marked by the tubulin marker DM1-α) are significantly more curved than spindles in control cell shown in the center of the upper panel of FIG. 2A.

In many cases only a fraction of the cells in a population exhibiting the rice phenotype will have bent spindles. Generally, between about 5 and 20 percent of the mitotic cells in such population will have bent spindles. More frequently, between about 9 and 15 percent of the mitotic cells will have bent spindles. These ranges have been typically found in SKOV cells having mitotic indexes of at least about 20%.

3. Alignment and Separation of Chromosomes During Mitosis

At relatively high doses of stimuli that produce the rice phenotype, chromosomes do not properly congress to the metaphase plate. During prometaphase, chromosomes in normal cells establish interactions with the fast-growing plus ends of microtubules via the kinetochore. The chromosomes then undergo a series of microtuble-dependent movements, culminating in alignment at the metaphase plate, equidistant from the two spindle poles. This process is called “congression.” In the phenotypes of this invention, chromosomes may appear to congress to metaphase, albeit in a delayed manner, and then fail to divide or maintain organization compared to control phenotypes. Generally, the DNA morphology is not static but does not follow the expected progression seen in the metaphase to anaphase transition of normal cells. In addition after prolonged arrest some DNA will spread out the spindle poles in a disorganized manner until the cell undergoes mitotic catastrophe or slippage into a G2 state.

The DNA aspect of the rice phenotypic may be observed by any technique that can distinguish chromosomal material from other cellular features and background. In many cases, it is convenient to generate images of cells that have been treated with markers for DNA and/or histones. Examples of such markers include fluorescently labeled antibodies to DNA and fluorescent DNA intercalators such DAPI and Hoechst 33342 (available from Molecular Probes, Inc. of Eugene, Oreg.) and antibodies to histones such as an antibody for a phosphorylated histone, e.g., phospho-histone 3 (pH3). As mentioned above, the histones in the nucleus become phosphorylated during mitosis and remain phosphorylated while the cell is in mitotic arrest. Therefore markers specific to phosphorylated histones will mark chromatin selectively in mitotic cells. Another option (although it does not selectively mark mitotic cells) is to use cells expressing a GFP-histone 2B (or any other GFP-tagged protein that functionally co-localizes with nuclear DNA).

In general the DNA aspects of the phenotype are not needed to assess the presence of the rice phenotype. To the extent that DNA or chromatin morphology is used to characterize phenotypes, automated image analysis can assess differences from control cells with respect to texture, size, and the long axis of an ellipse, etc. and thereby differentiate mitotic defects from control, and rice phenotype producing compounds (or other stimuli) from other compounds causing mitotic arrest.

In some embodiments, the chromosome or chromatin feature of the rice phenotype observed during mitosis can be presented as a multivariate signature. For example, this feature might be characterized by a signature combining the following values: (1) time from onset of mitosis to metaphase, (2) location of chromatin with respect to an expected metaphase plate during metaphase, and (3) failure to reach anaphase (Y or N). In this example, the resulting multivariate signature is characterized in terms of its “distance” (in multivariate phenotype space) from a control phenotype signature. Certain separation distances are associated with the rice phenotype of this invention. Various techniques for measuring distance in multivariate space may be used. Some are described below in the context of interphase phenotypes.

It can be useful to employ time-lapse imaging technology to characterize the progression of chromosomes during mitosis. As described above, the phenotypes of this invention are characterized by elongated spindles during mitotic arrest with dynamic, yet relatively unstructured, DNA movements and reorganizations. A specific example of a time-lapse experiment will now be described. Using multi-site time-lapse imaging of live cells expressing a GFP-histone 2B (or other GFP-tagged histone) at low (5×-10×) magnification, the mitotic DNA progression can be observed. Cells can be kept alive in their preferred environment using an environmental chamber with heat and carbon dioxide, using for example, apparatus available for this purpose such as the ImageXpress live cell imaging system available from Axon Instruments of Union City, Calif. Many wells can be sequentially visited and images can be taken. This process can be repeated every 10-15 minutes over a course of days, if appropriate, in the presence of a compound or control conditions, until hundreds of images are collected that can be collated into movies and analyzed qualitatively or quantitatively.

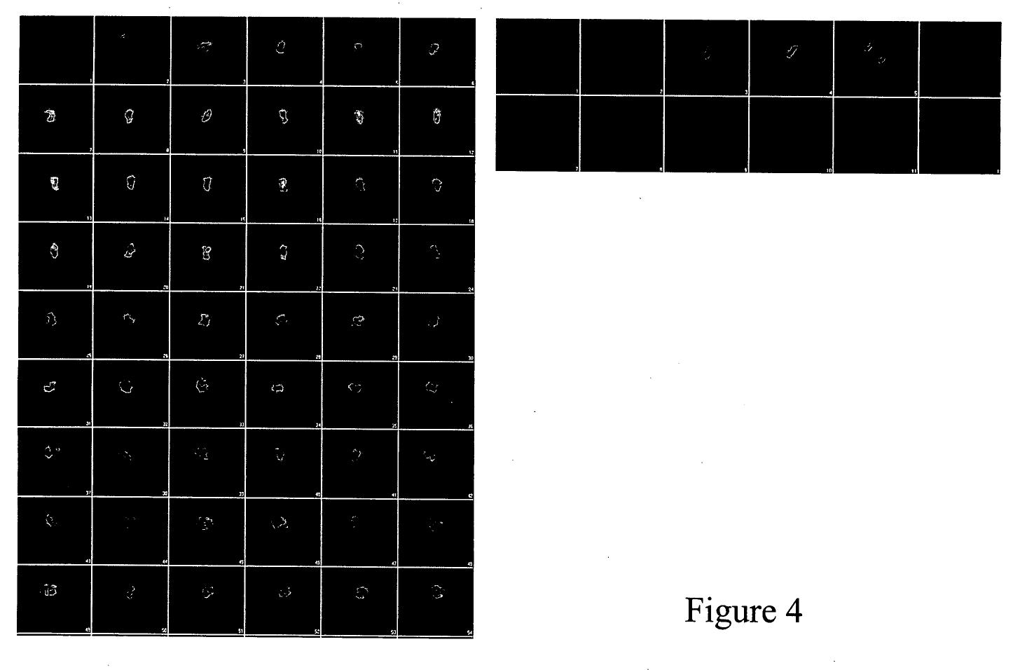

FIG. 4 shows timelapse montages of GFP-histone 2B in SKOV3 cells every 10 minutes, moving left to right in rows and then top to bottom. The left panel illustrates mitotic behavior of the histone 2B in a phenotype of this invention, while the right panel illustrates mitotic behavior in a control phenotype. The changing positions of the histones illustrate movement of chromatin during mitosis. The images of test cells in the left panel show the phenotypic response caused by treatment with 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester at 33 uM for 9 hours in one cell after the onset of mitotic arrest. The images of control cells shown in the right panel were treated with 0.4% DMSO during mitosis covering 2 hours.

As indicated, chromosomes in the rice phenotype appear to congress with a delayed metaphase but then fail to divide or maintain organization compared to control. The DNA morphology is dynamic and changes with time. This suggests that cells exhibiting the rice phenotype either fail to complete alignment or have a defect in the metaphase to anaphase transition. For comparison, the control montage shows normal mitotic progression of chromatin. In successive images numbered 2, 3, 4, 5, and 6, the control cell progresses from prophase (image 3), to prometaphase, to metaphase, to anaphase (image 5) and onto telophase (image 6). In the left panel, the rice phenotype cell has reached a “pseudo-metaphase” by the time when image 6 or 7 was reached. However, the chromatin never appears to segregate into daughter chromosomes.

As with most aspects of a phenotype, this feature of the phenotype is not significant at relatively low doses of the stimuli in question. But generally, it has been consistently observed in cases where the mitotic index is at least about 20%.

4. Normal Interphase Phenotype

Frequently, cells exhibiting the rice phenotype present unique features only during mitosis. During interphase, the phenotypic features of rice and control cells may be essentially indistinguishable. That is, only minimal phenotypic differences occur between control and rice phenotype cells during interphase, at least with respect to certain components of interest such as tubulin, DNA, and Golgi.

This behavior suggests that the target of the compounds that are eliciting the rice phenotype are specific for a protein or proteins that are only used by the cell in mitosis and are specific for those targets. This is similar to inhibition of the mitotic kinesin KSP. Other compounds that arrest cells in mitosis via targets that are also used during interphase (for example Taxol, Vincristine, and Vinblastine which target microtubules) show clear morphological effects on interphase and are predicted to have much lower therapeutic indexes in the human body.

Generally, in order to characterize the interphase phenotype of a cell or cell population, one must first determine whether a cell is in an interphase stage. As explained above, mitotic and interphase cells can be distinguished by analyzing various particular cellular features. For example, the signal from a marker for a phosphorylated histone may be used for this purpose. As indicated, one example of such marker is a marker for phospho-histone 3 (PH3) such an anti-phospho-histone 3 (PH3) antibody coupled to a fluorophore. If PH3 staining is not available, or desirable, then cells can be classified as mitotic or interphase based on a combination of the size of nuclei and the amount of DNA material in nuclei (as revealed by DNA staining using DAPI or Hoechst stains). After each cell, or image object, has been classified as interphase or mitotic, the mitotic and interphase phenotypes can be characterized.

The phenotype of the interphase cells may be characterized in terms of a wide variety of cellular features. Such features can relate to nuclear or cellular morphology, e.g., size, area, shape metrics, branching, etc. Cellular features relating to measures of the total amount of a component of a cell can be used, e.g. the total tubulin, total actin, total Golgi apparatus and other measures, often derived from measurements of the total intensity of radiation captured from a particular component of a cell. Also, measures of the texture of a cellular image can be used and which relate to physical properties of components of cells. Still other cellular features relating to various different types of generic cellular phenomena can be related to the interphase phenotype, such as changes in growth rate, cytoskeletal organization, alterations in organization and functioning of the endocytotic pathway, changes in expression and/or localization of transcription factors, receptors and the like. One, some or all of those cellular features can be considered in characterizing the interphase phenotype.

In one specific example, a particular group of cellular features for characterizing the interphase phenotype of a cell could include, for all cells that are not mitotic:
the average size of cell nuclei;
the average elliptical axis ratio for nuclei;
the average kurtosis intensity of cells;
the average pixel intensity for Golgi apparatus in cells;
the average cell area;
the elliptical axis ratio for cells;
the form factor (area divided by perimeter) for cells;
the kurtosis of the intensity of tubulin;
the second moment of a cell;
the average total intensity of tubulin for each cell;
the proportion of branched (i.e. having projections) cells.

In this example, the above group of cellular features constitutes the group of cellular features, which in combination define the interphase phenotype signature. A sub-group of these features can be used, or alternatively other groups of cellular features can be used. As will be appreciated, there are a large number of variables in this group of features. Some of these variables may be more important than others, i.e., may be more affected by a treatment than others. The combination of these features can be thought of as defining a vector in a multivariate space (defined by the cellular features) and which is characteristic of the interphase phenotype.

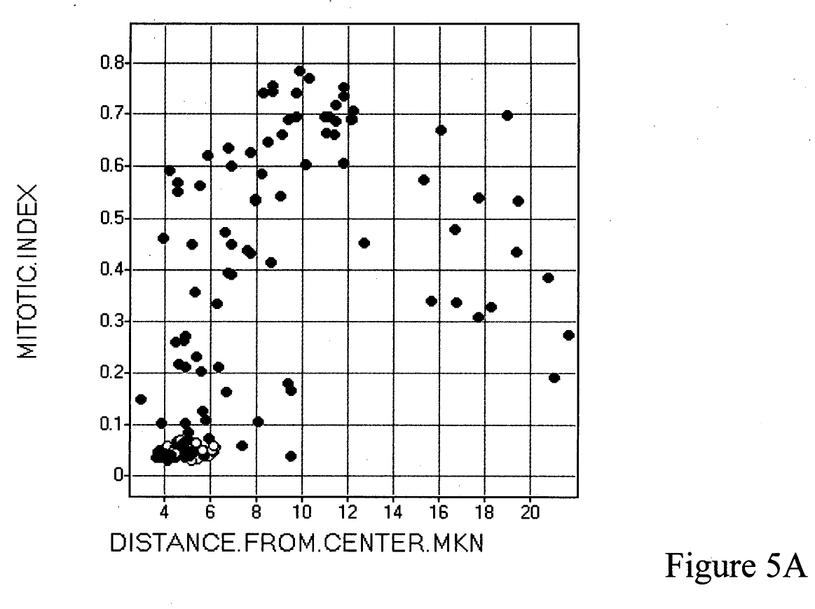

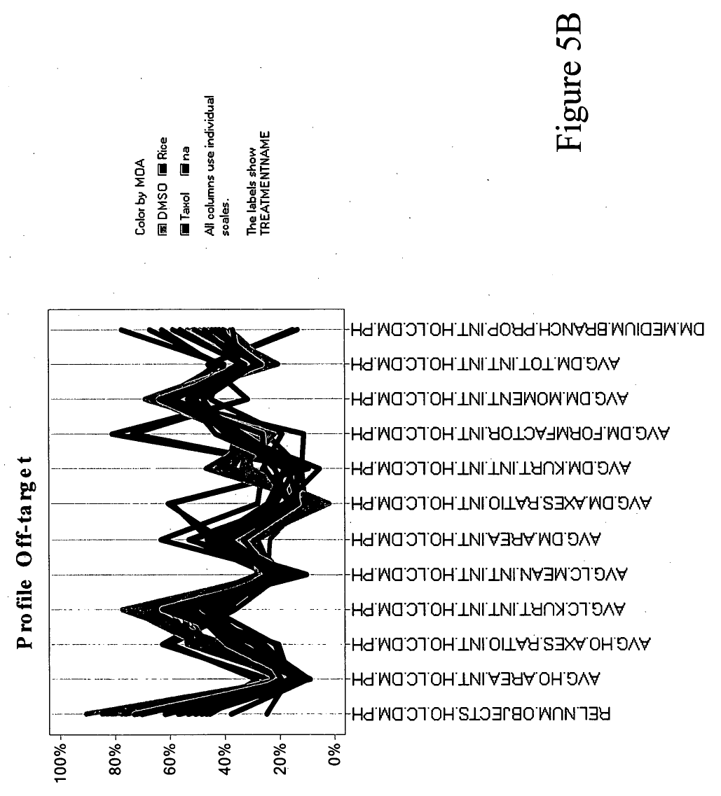

In one embodiment, after each cellular feature has been characterized, and similarly for the control group cellular features, a distance in multivariate space may be calculated. This can be the distance from a normal interphase phenotype as presented in the horizontal axis of FIG. 5A (described below). For the purposes of simplicity of discussion, if it is assumed that there are only three cellular features (a, b, c) comprising the interphase phenotype signature, and where the subscript ‘t’ refers to a feature of a treated cell and the subscript ‘c’ refers to a feature of a control cell, then the distance (L₁) in multivariate space between the interphase signature of the treated cells and interphase signature of the control cells can be calculated as L₁=|aₜ−a_{c}|+|bₜ−b_{c}|+|cₜ−c_{c}|, which provides the interphase metric.

Alternatively, the Euclidean distance (L₂) can be calculated using L₂=√((aₜ−a_{c})²+(bₜ−b_{c})²+(cₜ−c_{c})₂) to provide the interphase metric. Other methods of calculating the separation in multivariate space between the treated cell interphase signature and the control cell interphase signature can also be used. Note that any of the various methods described in this section may be employed to similarly measure distance between multivariate signatures of chromatin observed in mitotic cells that potentially exhibit the phenotypes of this invention.

In treatments other than those producing the rice phenotype, one may commonly observe, in the interphase cells, a breakdown of the actin cytoskeleton of a cell, or the Golgi apparatus. This breakdown may be a more or a less dominant effect of the treatment than mitotic breakdown. Regardless, such effects will result in a relatively large separation distance from the control phenotype for interphase cells.

FIG. 5A presents data showing that certain compounds producing the rice phenotype have very little effect on phenotypic features of interphase cells. In FIG. 5A, the vertical axis presents mitotic index, which is a measure of a compound's ability to cause mitotic arrest (thereby its ability to have a profound effect on the phenotype of mitotic cells), and the horizontal axis presents a “combined distance” from a normal interphase phenotype. The combined distance takes into account various features that characterize interphase phenotype, including those described above (i.e., the average size of cell nuclei, the average elliptical axis ratio for nuclei, the average kurtosis intensity of cells, etc.) Greater values on the horizontal axis indicate greater deviations from a control phenotype for interphase cells.

As explained, many stimuli that have a significant impact on mitosis also have some clearly defined impact on interphase features. This is exactly what is observed with a compound such as Taxol. Note that the Taxol data extends well into the region on the right side of the plot where the interphase phenotype is widely separated from the control interphase phenotype. However, the compounds that produce the rice phenotype (e.g., compounds 4-(3-Bromo-4-hydroxy-5-methoxy-phenyl)-1,6-dimethyl-2-oxo-1,2,3,4-tetrahydro-pyrimidine-5-carboxylic acid cyclopentyl ester and 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester in FIG. 5A) have minimal impact on interphase features—while having a significant impact on mitotic index. This is illustrated by the fact that all data points for these compounds lie on the left side of the plot in FIG. 5A. In this analysis, when the distance value is less than about 8 a compound is generally considered to have little effect on interphase cells.

FIG. 5B is a profile graph showing that the interphase cells treated with rice compounds track interphase control cells for various interphase phenotypic features. These features include, as shown on the horizontal axis, the relative number of objects (putative cells), the average area occupied by DNA in the objects (presumably nuclei), the average axes ratio of the DNA regions in the objects, the average kurtosis of the Golgi regions, the mean intensity of the Golgi regions, average area encompassed by tubulin in the objects, the average axes ratio of tubulin in the objects, the average kurtosis of the tubulin regions, the average form factor of the tubulin regions, the average moment of the tubulin regions, the average total intensity of tubulin in each object, and the branching properties of the tubulin in the objects. Note that the cells used to generate this graph were treated with markers for Golgi (LC for labeled LC Lectin), DNA (HO for Hoechst stain), and tubulin (DM for labeled DM1-α). As shown, the rice features closely track those of the DMSO treated cells and diverge strongly from the interphase cells of Taxol treated cells.

The data of FIG. 5B were obtained by treating SKOV3 cells with many different compounds and then capturing the interphase characteristics shown in the horizontal axis using image analysis techniques. The concentration of each compound was chosen by determining what concentration of a compound produced a mitotic index between 14 and 17% after cells were treated at multiple concentrations and then imaged to capture Golgi, tubulin, and DNA data for the interphase features shown in FIG. 5B. Each feature was scaled between 0 and 100% using the complete set of values for that feature obtained from all compounds under consideration. Only data for DMSO (control), and Taxol and the rice compound conditions that induced approximately 15% mitotic arrest are shown in FIG. 5B.

Compounds, concentrations, with mitotic index and off-target values shown in FIG. 5B are listed here in Table 1.

TABLE 1

| CONCEN- | | | | |
|---|---|---|---|---|
| TRATION | | | Mitotic | Interphase |
| (Molar) | TREATMENT | Class | Index | Off-Target |
| 4.00E−05 | 4-(3-Bromo-4- | Rice | 16% | 2.0 |
| | hydroxy-5-methoxy- | | | |
| | phenyl)-1,6-dimethyl- | | | |
| | 2-oxo-1,2,3,4- | | | |
| | tetrahydro-pyrimidine- | | | |
| | 5-carboxylic acid 3- | | | |
| | methyl-butyl ester | | | |
| 1.00E−05 | 4-(3-Bromo-4- | Rice | 17% | 0.6 |
| | hydroxy-5-methoxy- | | | |
| | phenyl)-6-methyl-2- | | | |
| | oxo-1,2,3,4- | | | |
| | tetrahydro-pyrimidine- | | | |
| | 5-carboxylic acid | | | |
| | 1,2,2-trimethyl-propyl | | | |
| | ester | | | |
| 2.00E−05 | 4-(3-Chloro-4- | Rice | 16% | 1.1 |
| | hydroxy-5-methoxy- | | | |
| | phenyl)-6-methyl-2- | | | |
| | oxo-1,2,3,4- | | | |
| | tetrahydro-pyrimidine- | | | |
| | 5-carboxylic acid tert- | | | |
| | butyl ester | | | |
| 4.00E−05 | 4-(4-Hydroxy-3-iodo- | Rice | 17% | 1.8 |
| | 5-methoxy-phenyl)-6- | | | |
| | methyl-2-oxo-1,2,3,4- | | | |
| | tetrahydro-pyrimidine- | | | |
| | 5-carboxylic acid tert- | | | |
| | butyl ester | | | |
| 2.00E−05 | Rename4-(3-Bromo- | Rice | 16% | 2.2 |
| | 4-hydroxy-5-methoxy- | | | |
| | phenyl)-1- | | | |
| | ethoxycarbonylmethyl- | | | |
| | 6-methyl-2-oxo- | | | |
| | 1,2,3,4-tetrahydro- | | | |
| | pyrimidine-5- | | | |
| | carboxylic acid tert- | | | |
| | butyl ester | | | |
| 2.00E−05 | 4-(4-Amino-3,5- | Rice | 16% | 1.1 |
| | dibromo-phenyl)-6- | | | |
| | methyl-2-oxo-1,2,3,4- | | | |
| | tetrahydro-pyrimidine- | | | |
| | 5-carboxylic acid tert- | | | |
| | butyl ester | | | |
| 4.00E−05 | 4-(3-Bromo-4- | Rice | 14% | 1.5 |
| | hydroxy-phenyl)-6- | | | |
| | methyl-2-oxo-1,2,3,4- | | | |
| | tetrahydro-pyrimidine- | | | |
| | 5-carboxylic acid tert- | | | |
| | butyl ester | | | |
| 6.25E−09 | Taxol | Micro- | 16% | 5.6 |
| | | tubules | | |
| 6.25E−09 | Taxol | Micro- | 17% | 5.4 |
| | | tubules | | |

As a specific example, see the interphase cells (flat round objects having generally uniform intensity) in the image of FIG. 6A. In this figure, the upper panel shows control cells treated with 0.4% DMSO for 24 hours and then imaged. The lower panel shows test cells treated with 4-(3-Bromo-4-hydroxy-5-methoxy-phenyl)-1,6-dimethyl-2-oxo-1,2,3,4-tetrahydro-pyrimidine-5-carboxylic acid cyclopentyl ester at 40 uM for 24 hours and then imaged. As shown in FIG. 6A, nuclear DNA (as marked by GFP-Histone) is similarly organized in rice interphase cells as it is in normal interphase cells.

As another example, see the interphase cells in the tubulin and DNA images of FIG. 6B. In that figure, the upper panels show tubulin (marked with DM1-α) and lower panels show DNA (marked with Hoechst 33342). The left panels show control cells treated with 0.4% DMSO for 24 hours and then imaged. The right panels show test cells treated with 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester at 10 uM for 24 hours and then imaged. One can see (in the circled regions for example) that at similar densities the control and rice interphase cells are similar in morphology.

5. Cell Line Specific Response to Stimuli that Induce the Phenotype

Interestingly, stimuli that induce the rice phenotype in some cell lines do not induce it other cell lines. For example, it has been found that compounds capable of inducing the rice phenotype in SKOV3 cells, DU145 cells, and SF268 cells (a CNS cancer line) fail to significantly induce the phenotype in A549 cells and HeLa cells at concentrations where the rice phenotype is achieved in other cell lines. Each of these cell lines (including those found not to exhibit the phenotype) is known to be sensitive to mitotic inhibitors. It may be that with more potent compounds these cell lines will exhibit the rice phenotype. It was also observed that a normal cell line HUVEC, which does not divide, is immune over a short time course. But it is apparently immune to all anti-mitotic agents.

The cell line specificity of the rice phenotype can be considered unique for its target. Many compounds that promote mitotic arrest also show cell line specificity, but at varying degrees for different cell types. The NCI measures the sensitivity of 60 cell lines to a wide panel of therapeutic agents and that data shows that compounds can be classified by the pattern of their sensitivity, and that a compound, like Taxol, can have over 3 orders of magnitude in potency differences between cell types. A compound can thus be uniquely described by its cell line specificity pattern, such that any compound with that pattern probably causes the same phenotype

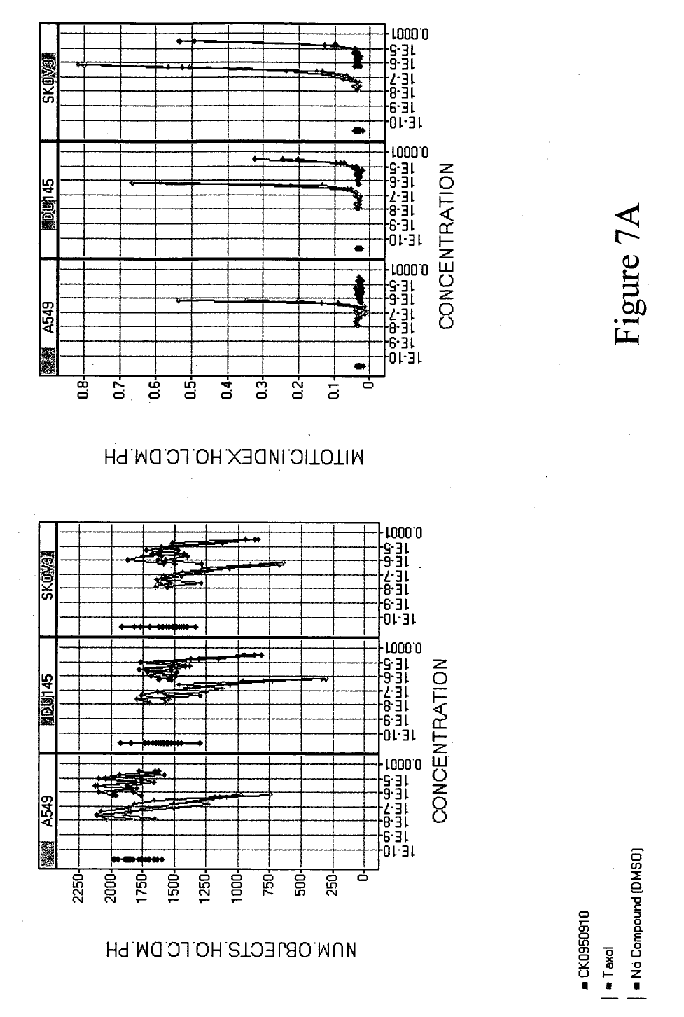

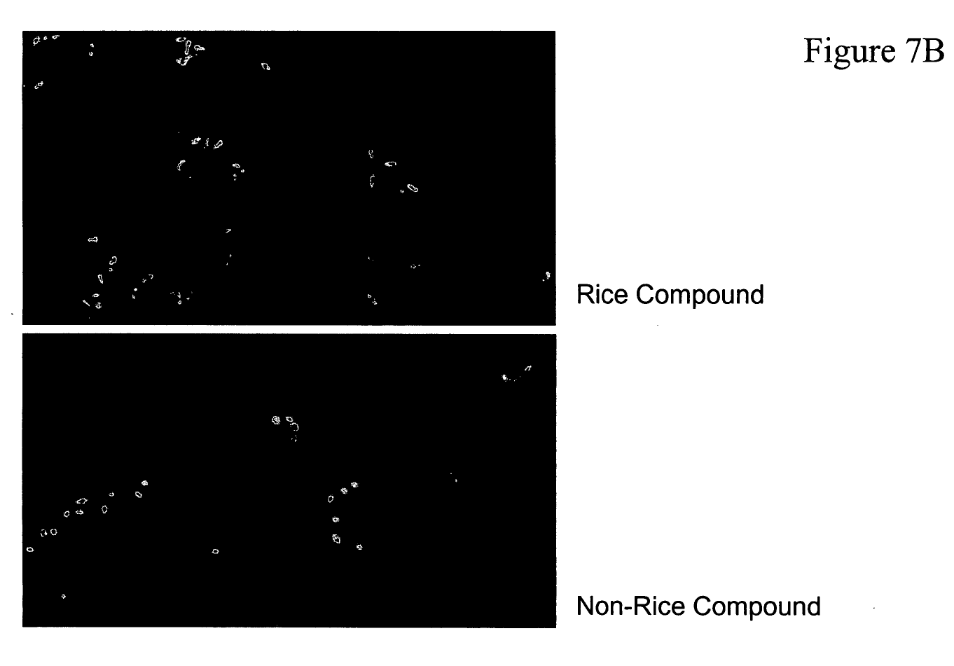

As an example, FIG. 7A shows that the compound 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester has a limited effect on mitotic index and cell number in A549 cells, while having significant affects on cell number and mitotic index in the DU145 and SK0V3 cell lines. As shown, the discrepancy exists over a wide range of concentrations. The two effects depicted in FIG. 7A are number of objects (a measure of the compound's cell killing strength) and mitotic index (fraction of the cells in a population that are in the mitotic phase). In the figure, compound 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester is compared to Taxol, which produces a robust response in A549 cells, and DMSO, which is inactive.

FIG. 7B compares phenotypes of DU145 cells that were treated with 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester (top panel) to phenotypes of DU145 cells that were treated with a different compound (bottom panel). The figure shows tubulin staining of the cells. As shown, 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester produces a rice phenotype, while the other compound does not. Note that the other compound produces a phenotype bearing some resemblance to the rice phenotype in SKOV3 cells. Specifically, it produces mitotic spindles that are somewhat elongated. In treatments with 4-(3-Bromo-4-hydroxy-5-methoxy-phenyl)-6-methyl-2-oxo-1,2,3,4-tetrahydro-pyrimidine-5-carboxylic acid 1,2,2-trimethyl-propyl ester the rice phenotype is clearly manifest in both SKOV3 cells and DU145 cells. This was not observed with the other compound. Hence, the other compound used in this study cannot be said to induce the rice phenotype.

6. Cells Die in a Defined Manner

Most cells exhibiting the rice phenotype ultimately die. Hence a population of cells exhibiting the phenotype will have, in comparison to a control, an unusually high proportion of cells that have died. Various techniques for identifying dead cells may be used. In the context of image analysis, cell count (or number of objects) is a useful measure of the impact of a stimulus on cell viability.

Cells exhibiting the rice phenotype typically die in one of two ways after a prolonged mitotic arrest. In both cases, the cells ultimately die by what is morphologically similar to an apoptotic or mitotic catastrophe pathway. In both cases, it is only mitotic cells that die. In one case, a cell progresses to apoptosis (or a morphologically similar state) directly from mitosis. In the other case, a cell first transitions to a state where its DNA decondenses, or slips back into a 4N state (four sets of chromosomes). From this state, it progress to apoptosis (or the similar state). In many cases, a population of rice phenotype cells will have significant numbers of cells dying by each mechanism (e.g., about 35% of the cells die via the decondensed DNA route and about 65% die via the direct route). These modes of cell death and the relative numbers of cells dying by these two modes are characteristics that may be employed to identify cells exhibiting the rice phenotype.

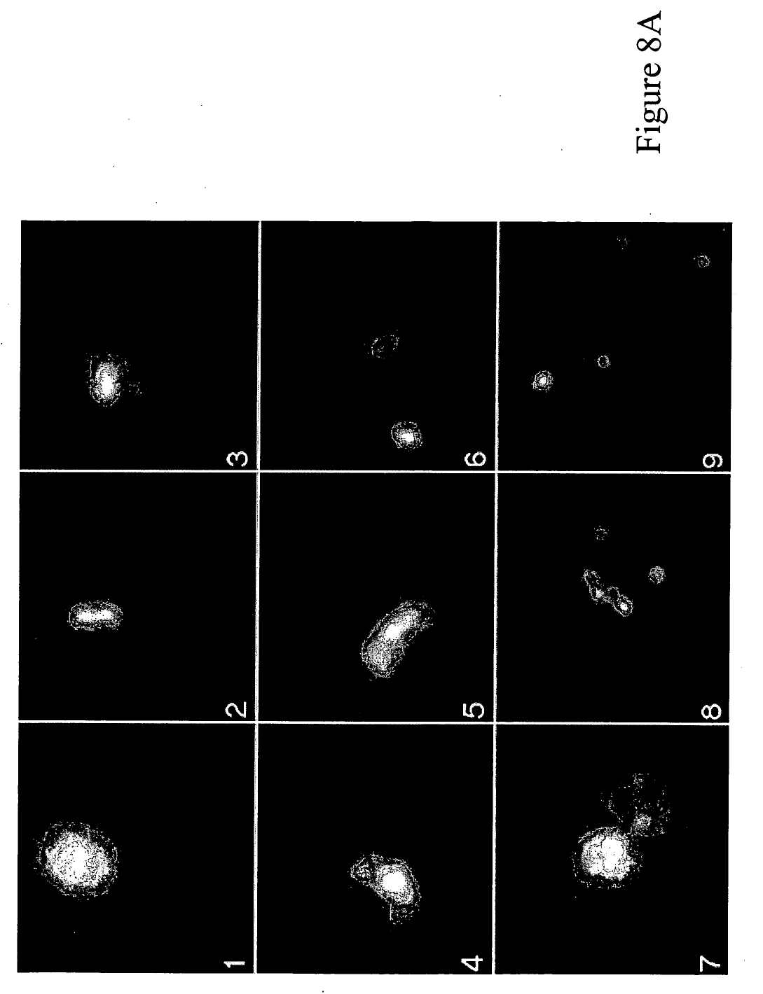

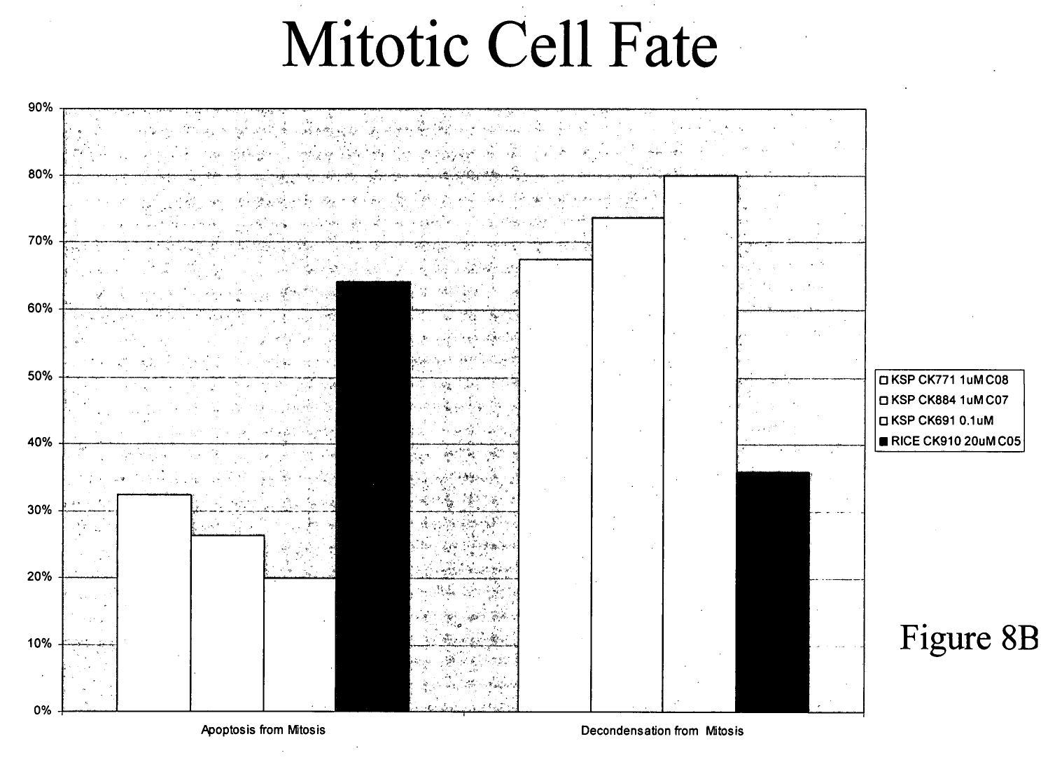

FIG. 8A shows a time-lapse montage of a SKOV3 cell exhibiting the rice phenotype undergoing DNA decondensation and then apparent apoptosis. The cells were marked with green fluorescent protein linked to histone 2B. Hence the images of FIG. 8A show the nucleus of the cell. Panel 1 shows cellular DNA during interphase. Panel 2 shows congression of DNA to a metaphase plate. Panels 3-5 show the DNA arrested in mitosis and unable to maintain the metaphase plate. By panel 5 the DNA has moved along the length of the elongated mitotic spindle (inferred). In panel 6 the DNA begins decondensing until it has formed 2 major clusters of DNA within one cell body. In panel 8 the decondensed DNA begins to fragment into multiple pieces (a signature of apoptosis). In panel 9 those DNA fragments scatter. This is one pathway leading to cell death after prolonged mitotic arrest. Some cells would not go through steps 6 and 7, but rather go directly to 8 from 5.

FIG. 8B is a bar chart showing (for rice and KSP phenotypes) the relative numbers of cells undergoing apparent apoptosis directly from mitosis and undergoing decondensation prior to death. The data used to construct the chart was obtained by time-lapse movies of SKOV3 cells marked with GFP-Histone 2B. In one experiment, movies were collected on cell populations treated with three different compounds that target that target KSP and one compound that induced the rice phenotype. As shown, cells exhibiting the rice phenotype have relatively more cells that die directly from mitosis and relatively fewer cells that die by first undergoing decondensation of their DNA.

To the extent that rice phenotype cells undergo apoptosis, various techniques may be employed to identify apoptotic cells. As illustrated with FIG. 8A, such cells can be identified visually as those that stop moving and whose nuclei fragment. More fundamentally, apoptosis is characterized by a pathway that includes changes in certain membrane proteins, depolarization of the mitochondrial membrane, release of cytochrome C from mitochondria, activation of various caspase enzymes (caspase 3 is a major isoform involved in apoptosis), condensation, fragmentation and granularization of the nuclei, and breakdown of various nuclear and cellular proteins including actin, and microtubules. In addition, apoptotic cells become loosely attached to their substrate and can float away. Many of these manifestations can be identified by image analysis. Examples include exposure of phosphatidyl serines on membrane proteins, the migration of cytochrome c from the mitrochondria into other regions of the cell, changes of mitochondrial membrane potential, activation of caspase 3, cleavage of caspase substrates (PARP, microtubule and actin), and condensation, fragmentation and granularization of the nuclei.

Another property of cells undergoing apoptosis is that they tend to become loosely attached to a substrate. Both cytoplasm shrinkage and loss of attachment is probably a result of cytoskeleton damage by caspases. This property can be detected by exposing the culture to a treatment that will tend to dislodge and remove loosely attached cells. One way to accomplish this is by carefully washing a cell culture under consideration. The level of apoptosis has been found to correlate well to a “washout coefficient” based on cell counts in washed and unwashed cultures exposed to a stimulus suspected of inducing apoptosis; e.g., (cc (unwashed)−cc(washed))/cc(unwashed).

A more detailed discussion of various techniques for identifying apoptotic cells is presented in U.S. patent application Ser. No. 10/623,486, filed Jul. 18, 2003, by Mattheakis et al., and titled “PREDICTING HEPATOTOXICITY USING CELL BASED ASSAYS,” and U.S. patent application Ser. No. 10/719,988, filed Nov. 20, 2003, by Mattheakis et al., and titled “PREDICTING HEPATOTOXICITY USING CELL BASED ASSAYS,” both of which are incorporated herein by reference in their entireties and for all purposes.

IV. Experimental Protocol

An experiment to determine whether a treatment can produce the rice phenotype can be carried out in many ways. Frequently it will involve one or more assay plates. An assay plate is typically a collection of wells arranged in an array with each well holding at least one cell or a related group or population of cells which have been exposed to a treatment or which provides a control group, population or sample. In other embodiments, multi-well plates are not used and single sample holders can be used. As explained above, a treatment can take many forms and in one embodiment can be a particular drug or any other external stimulus (or a combination of stimuli and/or drugs) to which cells are exposed on an assay plate or have previously been exposed. Experimental protocols for investigating the effect of a treatment will be apparent to a person of skill in the art and can include variations in the dose level, incubation time, cell type, cell line, marker set and other parameters, which are typically varied as part of an experimental protocol. After the cells have been treated, the extent of the effect of the treatment for producing the rice phenotype is evaluated by investigating, typically in a quantitative way, how the properties of the cells that are involved in or related to the rice phenotype have changed.

For example, the phenotypic feature of interest could be congression and alignment of chromosomes during mitosis. After the treatment has been applied to the cells and features have been extracted from captured images, then some of the cellular features can be used to classify cells as interphase or mitotic. As previously explained, the amount of fluorescence from an anti-phospho-histone 3 (PH3) coupled to a fluorophore can be used to distinguish between mitotic and interphase cells. After each cell, or image object, has been classified as interphase or mitotic (or discarded as being an imaging artefact), a characterization of mitotic chromatin can be made. The effect of the treatment can then be determined by comparing this characterization for the treated cells with the same characterization for a control group of cells.

As explained, there will likely be other cellular features of cell components which are involved in or relate to rice phenotype and which will also be affected by the treatment and so change. Therefore using a one or a combination of the relevant phenotypic features, the effect of the treatment can be evaluated.

In addition to merely determining whether a given treatment produces the rice phenotype of this invention, the investigation may study different dose levels of the stimulus (or stimuli) in question. It has been found that different dose levels and experimental protocols can result in different relevant phenotypic features arising. Significantly different dose levels may be required to produce the rice phenotypic features in different cell lines.

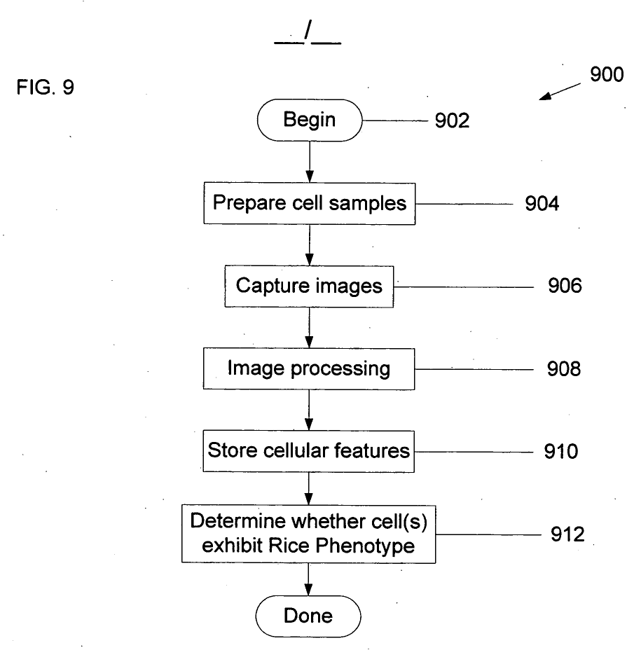

Having discussed the overall methodology of the invention, an example embodiment will now be described in greater detail in the context of an image based collection of cellular features. FIG. 9 shows a flow-chart 900 illustrating an example of the general method and illustrating various aspects of the invention. The method begins at 902 and at a step 904 cell samples are prepared for investigation.

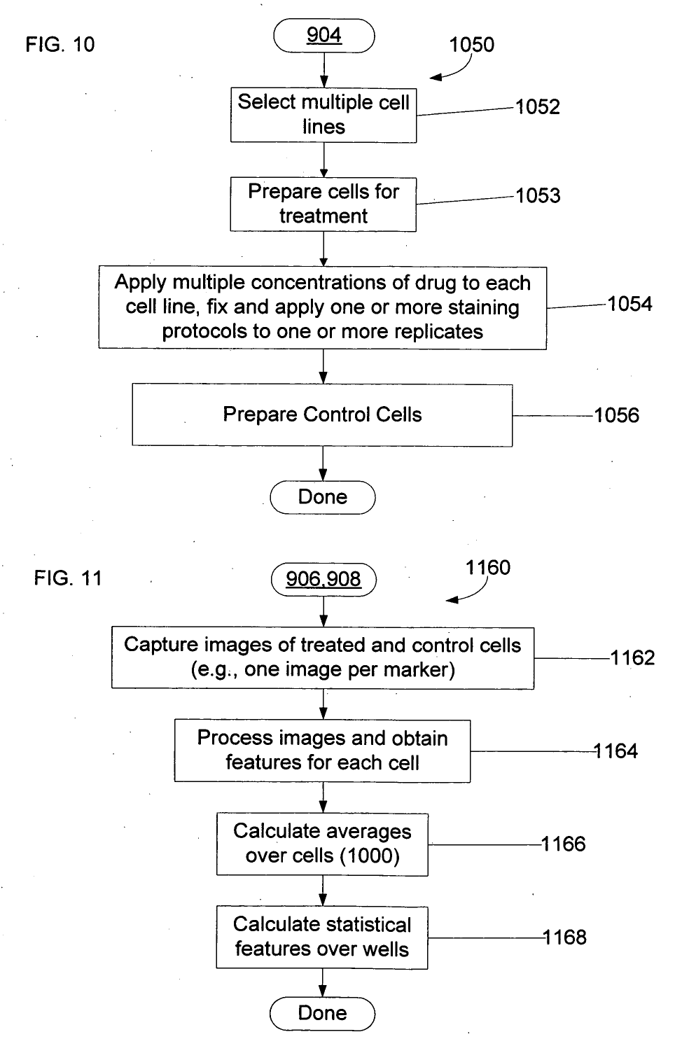

FIG. 10 shows a flow chart 1050 illustrating a number of cell sample preparation steps that can be carried out in one embodiment, giving an example of one suitable experimental protocol, and corresponding generally to step 904. Not all the activities and operations illustrated in FIG. 10 are essential. Some operations may be omitted and other operations may be added. The details of each operation may be varied depending on the particular experiment being carried out.

Although illustrated as sequential in FIG. 10, steps 1054 and 1056 do not need to be carried out in sequence and can be carried out in parallel, independently of each other. In a first step 1052, a particular one or a plurality of different cell types are selected. In the embodiment described, six cell lines for the particular cell type are selected although fewer or more cell lines can be used. In one embodiment, the cell lines used are A549, DU145, SKOV3 A498, HUVEC and SF268. Next, in a step 1053, the cells are prepared by, for example, plating them on appropriate substrates. At a step 1054, the treatment is applied to the cells. Well plates can be used to hold the cells and a population of cells from a single cell line is provided in each separate well arranged over a well plate or a number of well plates.

In the illustrated embodiment, at step 1054, the cells are treated, chemically fixed, and stained. However, this is not necessary and in another embodiment, live cells can be used which express a fluorescent protein or stained with live dyes and so no fixing or staining operations are required. In greater detail, wells are provided holding a population of cells. The treatment, in this example a compound, to be investigated is applied to the cells at different concentration levels, by dilution in culture medium. In one example, eight different concentration or dose levels are used, with a different dose level in each well. Fewer or more dose levels can be used as appropriate. The experiment is replicated three times so as to provide three sets of results for each concentration level. Fewer replicates can be used based on cost considerations, but larger numbers of replicates are preferred as providing data with a lower noise level. The drug and cells can be allowed to incubate for a fixed period of time, e.g. in one embodiment 24 hours, to allow the treatment to take effect. In other embodiments, the cells are allowed to incubate for varying periods of time, in order to investigate the time variation of the treatment. The cells can then be chemically fixed, for a single time point assay. The cells for each cell line are subject to a first staining protocol and a second staining protocol, which may involve multiple stains depending on the number and type of cellular features to be marked. Hence, in the described embodiment, 288 wells (eight dose levels, six cell lines, two staining protocols and three replicates) are used each holding a cellular population or group therein.

At the same time as the treated cells are being prepared, a number of control populations of cells are also prepared in step 1056. Preparation techniques for control cells will be different depending on the drug formulation. The cells are subject to the same staining treatments, fixation and incubation periods as the treated cells, but without being subjected to the treatment. In one embodiment, the cells are incubated with DMSO, at the same percentage levels as that used to administer the treatments, in order to provide controls for each cell line and staining or experimental condition. In one embodiment eight control wells are provided on each well plate. This provides at least one control for each cell line/staining protocol combination. Hence the cell sample preparation step 904 results in eight treatment concentrations, in triplicate, with cells stained according to two different protocols, and for six different cell lines and with control populations of cells which have not been exposed to the treatment. It is not necessary to use more than one stain or staining protocol and in other embodiments a single stain only can be used.

Returning to FIG. 9, the cellular features can be obtained from the cells using an image capture and processing technique. At step 906, images of the cells are captured and at step 908 various imaging processing operations are carried out and cellular features are derived from the captured images of the cells. Once all the desired the cellular features have been obtained from the images, or derived from other cellular features, then the cellular features are stored for future use in the evaluation of the rice phenotype at a step 910. In another embodiment, the cellular features are used straight away to determine whether the rice phenotype has been produced and then discarded. In another embodiment steps 908 and 910 are bypassed and the images are manually evaluated. In other words, the rice phenotype can be identified qualitatively without steps 908 and 910

FIG. 11 shows a flow chart 1160 illustrating the image capture 906, processing and feature extraction 908 steps of flow chart 900 in greater detail. At a first step 1162, images of the cell populations in each well are captured. In this example, images are captured for each of the eight concentration levels, in triplicate for each cell line and for both of the staining protocols. Similarly, images are captured for each of the groups of control cells for each cell line and for both staining protocols. In particular, a first image or set of images is captured of each well for the stains used in the first staining protocol and then a second image or group of images for each well is captured for the stains used in the second staining protocol. One or more images can be captured for each well and/or each stain.

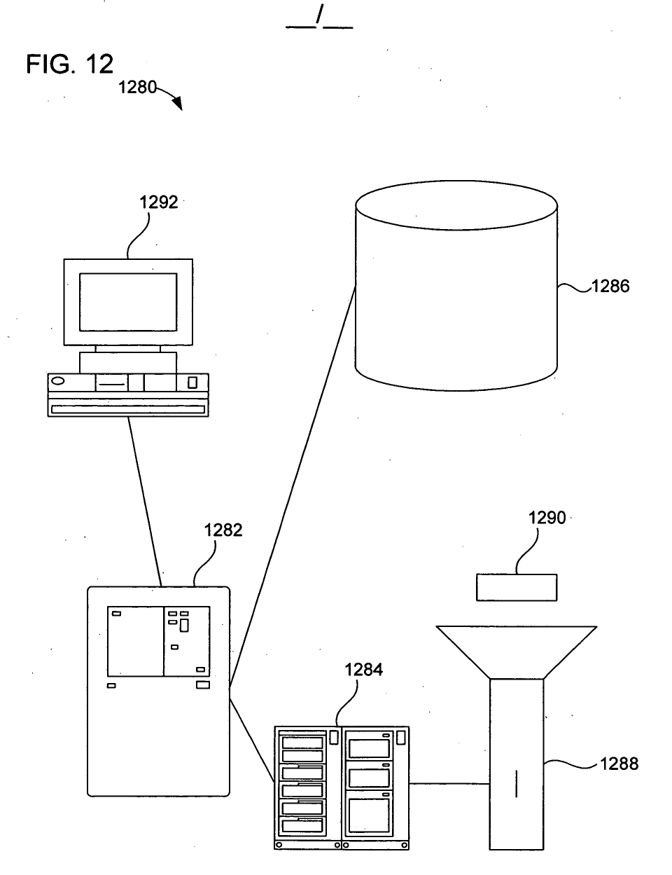

FIG. 12 shows a schematic block diagram of an image capture and image processing system 1280 which can be used to capture and process the images of cells or cell parts during steps 906 and 908 and store the cellular features in step 910. This diagram is merely an example and should not limit the scope of the claims herein. One of ordinary skill in the art would recognize other variations, modifications, and alternatives. The present system 1280 includes a variety of elements such as a computing device 1282, which is coupled to an image processor 1284 and is coupled to a database 1286. The image processor receives information from an image-capturing device 1288 which includes an optical device for magnifying images of cells, such as a microscope. The image processor and image-capturing device can collectively be referred to as the imaging system herein. The image-capturing device obtains information from a plate 1290, which includes a plurality wells providing sites for groups of cells. These cells can be cells that are living, fixed, cell fractions, cells in a tissue, and the like. The computing device 1282 retrieves the information, which has been digitized, from the image-processing device and stores such information into the database 1286.

A user interface device 1292, which can be a personal computer, a work station, a network computer, a personal digital assistant, or the like, is coupled to the computing device. In the case of cells treated with a fluorescent marker, a collection of such cells is illuminated with light at an excitation frequency from a suitable light source (not shown). A detector part of the image-capturing device is tuned to collect light at an emission frequency. The collected light is used to generate an image, which highlights regions of high marker concentration.

Sometimes corrections can be made to the measured intensity. This is because the absolute magnitude of intensity can vary from image to image due to changes in the staining and/or image acquisition procedure and/or apparatus. Specific optical aberrations can be introduced by various image collection components such as lenses, filters, beam splitters, polarizers, etc. Other sources of variability may be introduced by an excitation light source, a broadband light source for optical microscopy, a detector's detection characteristics, etc. Even different areas of the same image may have different characteristics. For example, some optical elements do not provide a “flat field.” As a result, pixels near the center of the image have their intensities exaggerated in comparison to pixels at the edges of the image. A correction algorithm may be applied to compensate for this effect. Such algorithms can be developed for particular optical systems and parameter sets employed using those imaging systems. One simply needs to know the response of the systems under a given set of acquisition parameters.

After the images have been captured, at step 1164, the captured images are processed using any suitable image processing and image correction techniques in order to extract the cellular features for the cells from the stored captured images.

A number of image processing steps can be carried out in step 1164 and not all the steps described are essential. Certain steps may be omitted and other steps may be added depending on the exact nature of the image capture process and markers used. The image can be corrected to remove any artefacts introduced by the image capture system and to remove any background. Other conventional image correction technique which will improve the quality of the image can also be used. Typically, one chooses nuclear markers and cytoplasmic markers which generate radiation at different wavelengths and so separate nuclear images and cytoplasmic images may be captured. Therefore different image correction techniques may be used for the nuclear and cytoplasm images, or for images captured of different markers or stains. Similarly, in the rest of the processes, different techniques may be used for the nuclear and cytoplasmic images, depending on the markers used. Also, different processing techniques can be carried out depending on the type of imaging that is used, e.g. brightfield, confocal or deconvolution.

After image correction, a segmentation process is carried out on the images in order to identify individual objects or entities within the image. Any suitable segmentation process may be used in order to obtain various cellular objects or components, such as nuclear and cellular objects and components. Typically nuclear DNA markers provide a strong signal and there is a high contrast in the image and an edge detection based segmentation process can be used. For segmenting cells, a watershed type method can be used instead. The segmentation process typically identifies edges where there is a sudden change in intensity of the cells in the image and then looks for closed connected edges in order to identify an object. Segmentation will not be described in greater detail as it is well understood in the art and so as not to obscure the present invention. As indicated above, exemplary segmentation procedures are described in U.S. Patent Publications Nos. US-2002-0141631-A1 and US-2002-0154798-A1.

Additional operations may be performed prior to, during, or after the imaging operation 906 of FIG. 9. For example, “quality control algorithms” may be employed to discard image data based on, for example, poor exposure, focus failures, foreign objects, and other imaging failures. Generally, problem images can be identified by abnormal intensities and/or spatial statistics.

In a specific embodiment, a correction algorithm may be applied prior to segmentation to correct for changing light conditions, positions of wells, etc. In one example, a noise reduction technique such as median filtering is employed. Then a correction for spatial differences in intensity may be employed. In one example, the spatial correction comprises a separate model for each image (or group of images). These models may be generated by separately summing or averaging all pixel values in the x-direction for each value of y and then separately summing or averaging all pixel values in the y direction for each value of x. In this manner, a parabolic set of correction values is generated for the image or images under consideration. Applying the correction values to the image adjusts for optical system non-linearities, mis-positioning of wells during imaging, etc.

Generally the images used as the starting point for the methods of this invention are obtained from cells that have been specially treated and/or imaged under conditions that contrast the cell's marked components from other cellular components and the background of the image. Typically, the cells are fixed and then treated with a material that binds to the components of interest and shows up in an image (i.e., the marker).

At every combination of dose, cell line and staining protocol, one or more images can be obtained. As mentioned, these images are used to extract various parameter values of cellular features of relevance to a biological, phenomenon of interest. Generally a given image of a cell, as represented by one or more markers, can be analyzed, in isolation or in combination with other images of the same cell (as provided by different markers), to obtain any number of image features. These features are typically statistical or morphological in nature. The statistical features typically pertain to a concentration or intensity distribution or histogram.

The various phenotypic features of the rice phenotype have been described above, together with techniques for identifying these features. The image analysis methods of this invention identify such features and possibly others. Some general feature types suitable for detection or quantification with this invention include a cell, or nucleus where appropriate, count, an area, a perimeter, a length, a breadth, a fiber length, a fiber breadth, a shape factor, a elliptical form factor, an inner radius, an outer radius, a mean radius, an equivalent radius, an equivalent sphere volume, an equivalent prolate volume, an equivalent oblate volume, an equivalent sphere surface area, an average intensity, a total intensity, an optical density, a radial dispersion, and a texture difference. These features can be average or standard deviation values, or frequency statistics from the parameters collected across a population of cells. In some embodiments, the features include features from different cell portions or cell lines.

After the features have been extracted from the image (1164) they are stored (910) in database 1286, and analysis of the features is carried out in order to assess the effect of the treatment on the cells.

As explained above, some of the cellular features obtained for the cells are simple features, e.g. the area of a nucleus. Other cellular features are statistical in nature, e.g. the standard deviation of the nuclear area for a group of cells, and reflect properties of the group of cells in a well or related wells. It will be appreciated that any simple or complex cellular feature than can be derived from the images is suitable for use in the present invention and that the invention is not to be limited to the specific examples given, nor to the specific sequence of actions, which is merely by way of an illustrative example. The result of step 1164 can be thousands or tens of thousands of cellular features derived from each of the treated wells and control wells.

In general in steps 1166 and 1168 cells from a well are evaluated and some statistics for that well, e.g. the average of a property, are calculated. Then, the same quantity is obtained for the replicate wells (e.g., the other five wells when the experiment is replicated six times) statistics are computed on those statistics for the replicate wells in order to aggregate (e.g., obtain the median of the average value mentioned above). However, averaging is not necessary and instead cell level information can be used, and have all further computations to be based on cell level information. Hence, for each compound/cell line/time point/marker set/etc there would be thousands of data points.

At step 1166, at each dose level and for each cell line, the cellular features can be averaged, e.g. to obtain an average nuclear area for the cells from a certain cell line at a certain dose level. Hence an average simple cellular feature can be obtained for each cell line at each dose level. However, it is not necessary to calculate averages over cells. Also, other statistical measures can be used such as the median, specific quantiles, standard deviations and other measures of the statistical properties of a group of objects. Further, the statistical properties need not be calculated over all cells, but can be calculated over a sub-population of cells, for example over the sub-group of interphase cells. In that case, a cell cycle related classification of the cells is carried out prior to summarizing or averaging the cell feature values.

At step 1168, more complex cellular features, based on a statistical analysis of the properties of the cells in the wells, rather than the properties of a single cell, are calculated over all the wells for each cell line at each dose level. Hence the cellular features obtained characterise the simple cellular features and statistical cellular features for the cellular populations at each dose level for each cell line.

In other embodiments, the simple cellular features and the statistical cellular features can be determined across cell lines so as to be characteristic of the effect of the treatment across different cell lines. In other embodiments, different incubation times can be used for a given concentration and the cellular features can be averaged over the different incubation times in order to provide cellular features characteristic of the effect of the treatment at the same dose level but over different incubation times.

Returning to FIG. 9, after the cellular features have been calculated and stored, at step 910 a quantitative measure of the presence or absence of the rice phenotype may be calculated based on the cellular features. See step 912.

Some embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer (e.g., computer 1282). The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium. Examples of program instructions include both machine code, such as produced by a compiler, and files containing higher level code that may be executed by the computer using an interpreter.

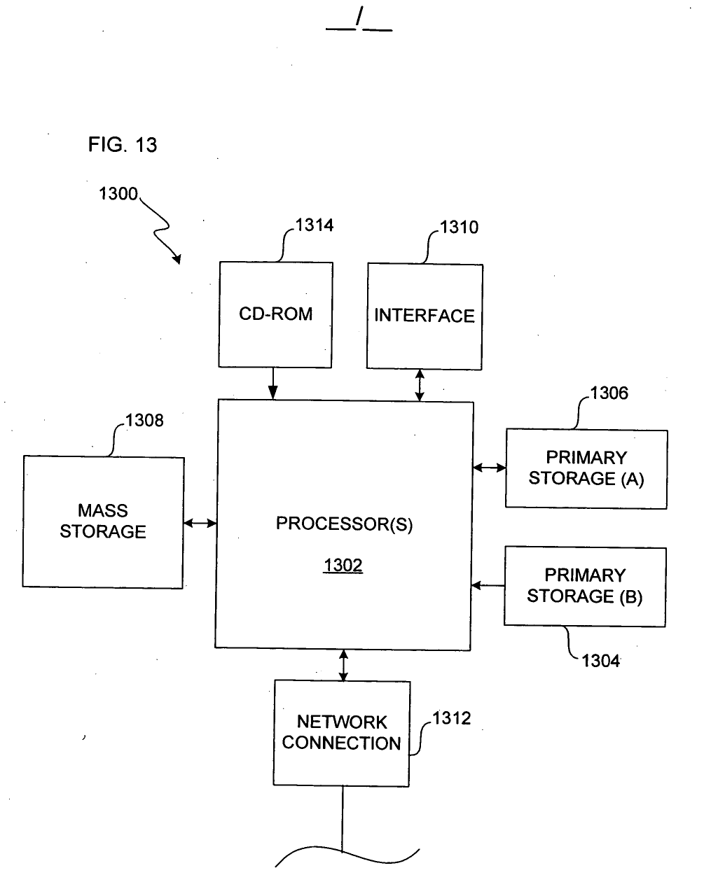

FIG. 13 illustrates a typical computer system that, when appropriately configured or designed, can serve as an image analysis apparatus of this invention. The computer system 1300 includes any number of processors 1302 (also referred to as central processing units, or CPUs) that are coupled to storage devices including primary storage 1306 (typically a random access memory, or RAM), primary storage 1304 (typically a read only memory, or ROM). CPU 1302 may be of various types including microcontrollers and microprocessors such as programmable devices (e.g., CPLDs and FPGAs) and unprogrammable devices such as gate array ASICs or general purpose microprocessors. As is well known in the art, primary storage 1304 acts to transfer data and instructions uni-directionally to the CPU and primary storage 1306 is used typically to transfer data and instructions in a bi-directional manner. Both of these primary storage devices may include any suitable computer-readable media such as those described above. A mass storage device 1308 is also coupled bi-directionally to CPU 1302 and provides additional data storage capacity and may include any of the computer-readable media described above. Mass storage device 1308 may be used to store programs, data and the like and is typically a secondary storage medium such as a hard disk. It will be appreciated that the information retained within the mass storage device 1308, may, in appropriate cases, be incorporated in standard fashion as part of primary storage 1306 as virtual memory. A specific mass storage device such as a CD-ROM 1314 may also pass data uni-directionally to the CPU.

CPU 1302 is also coupled to an interface 1310 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1302 optionally may be coupled to an external device such as a database or a computer or telecommunications network using an external connection as shown generally at 1312. With such a connection, it is contemplated that the CPU might receive information from the network, or might output information to the network in the course of performing the method steps described herein.

In one embodiment, the computer system 1300 is directly coupled to an image acquisition system such as an optical imaging system that captures images of cells. Digital images from the image generating system are provided via interface 1312 for image analysis by system 1300. Alternatively, the images processed by system 1300 are provided from an image storage source such as a database or other repository of cell images. Again, the images are provided via interface 1312. Once in the image analysis apparatus 1300, a memory device such as primary storage 1306 or mass storage 1308 buffers or stores, at least temporarily, digital images of the cells. In addition, the memory device may store the quantitative phenotypes that represent the points on the response path. The memory may also store various routines and/or programs for analyzing the presenting the data, including the phenotype characterization and image presentation. Such programs/routines may include programs for performing principal component analysis, regression analyses, path comparisons, and for graphically presenting the response paths.

VI. Other Embodiments

Although the above has generally described the present invention according to specific processes and apparatus, the present invention has a much broader range of applicability. In particular, the present invention has been described in terms of cellular phenotypes that are derived primarily from image analysis, but is not so limited, as the phenotypic characterizations presented herein may also be derived in whole or in part by techniques other than image analysis. Of course, those of ordinary skill in the art will recognize other variations, modifications, and alternatives.

1. A rice phenotype embodied in a mammalian cell or a population of mammalian cells, wherein the rice phenotype comprises:
(a) chromosomes that approach metaphase but fail to separate and maintain alignment compared to a control cell or cell population;
(b) a bipolar spindle that is at least about 10% longer than a corresponding metaphase bipolar spindle from the control cell or cell population; and
(c) during interphase, the cell or population of cells exhibits a phenotype that is substantially similar to that of an interphase control cell or the interphase cells of a control cell population.

2. The phenotype of claim 1, wherein the rice phenotype further comprises, in some cells in the population of cells, a metaphase mitotic spindle that is bent.

3. The phenotype of claim 2, wherein at least about 5% of the cells in the mitotic population of cells has a bent mitotic spindle.

4. The phenotype of claim 1, wherein the rice phenotype further comprises a higher percentage of the cells in the cell population that die prematurely in comparison to the control cell or cell population.

5. The phenotype of claim 1, wherein the cell or cells in the population of cells die by apoptosis upon reaching a mitotic state.

6. The phenotype of claim 5, wherein some of the cells that die by apoptosis do so after their DNA decondenses.

7. The phenotype of claim 1, wherein stimuli that produce the rice phenotype do so selectively in some cell lines and to a significantly lesser degree in others.

8. The phenotype of claim 1, wherein A549 cells are less susceptible to stimuli that produce the rice phenotype than DU145 and SKOV3 cells.

9. The phenotype of claim 8, wherein the DU145 cells are less susceptible to stimuli that produce the rice phenotype than the SKOV3 cells.

10. The phenotype of claim 1, wherein, during interphase, the rice phenotype is substantially similar to the control phenotype in terms of one or more of the following: cytoskeletal organization, cell shape, alterations in organization and functioning of the endocytic pathway, and changes in expression or localization of transcription factors or receptors.

11. A mammalian cell or mammalian cell population having a rice phenotype, wherein the rice phenotype comprises:
(a) chromosomes that approach metaphase but fail to separate and maintain alignment compared to a control cell or cell population;
(b) a bipolar spindle that is at least about 10% longer than a corresponding metaphase bipolar spindle from the control cell or cell population; and
(c) during interphase, the cell or population of cells exhibits a phenotype that is substantially similar to that of an interphase control cell or the interphase cells of a control cell population.

12. The cell or population of claim 11, wherein the rice phenotype is produced by applying a stimulus to the mammalian cell or cell population while the cell or cell population does not exhibit the rice phenotype in order to induce a transformation to produce the rice phenotype.

13. The cell or population of claim 12, wherein applying the stimulus comprises administering a compound to the mammalian cell or cell population while the cell or cell population does not exhibit the rice phenotype.

14. The cell or population of claim 11, wherein the rice phenotype further comprises, in some cells in the population of cells, a bipolar spindle that is bent.

15. The cell or population of claim 11, wherein the rice phenotype further comprises a higher percentage of the cells in the cell population that die in comparison to the control cell or cell population.

16. The cell or population of claim 11, wherein compounds that produce the rice phenotype do so selectively in some cell lines and to a significantly lesser degree in others.

17. A method of determining whether a compound produces a transformation associated with a rice phenotype, the method comprising:
(a) exposing a mammalian cell or mammalian cell population to the compound;
(b) allowing the compound to interact with the cell or cell population in a manner that transforms a normal phenotype in susceptible cells to the rice phenotype, wherein the rice phenotype has at least the following features:
(i) chromosomes that approach metaphase but fail to separate and maintain alignment compared to a control cell or cell population;
(ii) a bipolar spindle that is at least about 10% longer than a corresponding metaphase mitotic spindle from the control cell or cell population; and
(iii) during interphase the cell or population of cells exhibits a phenotype that is substantially similar to that of an interphase control cell or the interphase cells of a control cell population;
(c) imaging the cell or cell population to capture features that characterize the phenotype of the cell or cell population; and
(d) analyzing the image to determine whether the cell or cell population exhibits the phenotypic features specified in (b), to thereby determine whether the compound produces the transformation.

18. The method of claim 17, wherein the stimulus is a chemical compound.

19. The method of claim 17, wherein imaging the cell or cell population comprises capturing multiple images in a time-lapse manner.

20. The method of claim 17, wherein the rice phenotype further comprises, in some cells in the population of cells, a metaphase mitotic spindle that is bent.

21. The method of claim 17, wherein the rice phenotype further comprises a higher percentage of the cells in the cell population that die in comparison to the control cell or cell population.

22. The method of claim 17, wherein compounds that produce the rice phenotype do so selectively in some cell lines and to a significantly lesser degree in others.

23. The method of claim 17, wherein A549 cells are less susceptible to stimuli that produce the rice phenotype than DU145 and SKOV3 cells.

24. The method of claim 23, wherein the DU145 cells are less susceptible to stimuli that produce the rice phenotype than the SKOV3 cells.

25. The method of claim 17, wherein the bipolar spindle is at least about 15% than a corresponding metaphase mitotic spindle from a control cell or cell population.

26. A method of characterizing a mammalian cell or a mammalian cell population on the basis of its phenotype, the method comprising:
(a) receiving data characterizing the phenotype of the cell or cell population;
(b) analyzing the data to determine whether the cell or cell population possesses the following features:
(i) chromosomes that approach metaphase but fail to separate and maintain alignment compared to a control cell or cell population;
(ii) a bipolar spindle that is at least about 10% longer than a corresponding metaphase mitotic spindle from the control cell or cell population; and
(iii) during interphase the cell or population of cells exhibits a phenotype that is substantially similar to that of an interphase control cell or the interphase cells of a control cell population; and
(c) characterizing the cell or cell population as having a rice phenotype when the cell or cell population is found to possess at least the features specified in (b).

27. The method of claim 26, wherein the data characterizing the phenotype of the cell or cell population comprises data specifying whether the cell or cell population has been exposed to a stimulus that interacts with a target associated with the rice phenotype.

28. The method of claim 26, wherein (b) further comprises analyzing the data to determine whether the cell or cell population possesses the following additional features:
a bipolar spindle that is bent in some cells in the population of cells;
a higher percentage of the cells in the cell population that die in comparison to the control cell or cell population; and
stimuli that produce the rice phenotype do so selectively in some cell lines and not in others.

29. The method of claim 28, wherein at least about 5% of the cells in the population of cells has a bent mitotic spindle.

30. The method of claim 28, wherein the cell or cells in the population of cells die by apoptosis upon reaching a mitotic state.

31. The method of claim 30, wherein some of the cells that die by apoptosis do so after their DNA decondenses.

32. A computer program product comprising a machine readable medium on which is provided program code for characterizing a mammalian cell or a mammalian cell population on the basis of its phenotype, the program code comprising:
(a) code for receiving data characterizing the phenotype of the cell or cell population;
(b) code for analyzing the data to determine whether the cell or cell population possesses the following features:
(i) chromosomes that approach metaphase but fail to separate and maintain alignment compared to a control cell or cell population;
(ii) a bipolar spindle that is at least about 10% longer than a corresponding metaphase mitotic spindle from the control cell or cell population; and
(iii) during interphase the cell or population of the interphase cells exhibits a phenotype that is substantially similar to that of an interphase control cell or the interphase cells of a control cell population; and
(c) code for characterizing the cell or cell population as having a rice phenotype when the cell or cell population is found to possess at least the features specified in (b).

33. The computer program product of claim 32, wherein (b) further comprises code for analyzing the data to determine whether the cell or cell population possesses the following additional features:
a bipolar spindle that is bent in some cells in the population of cells;
a higher percentage of the cells in the cell population that die in comparison to the control cell or cell population; and
stimuli that produce the rice phenotype do so selectively in some cell lines and not in others.

34. An apparatus for characterizing a mammalian cell or a mammalian cell population on the basis of its phenotype, the apparatus comprising:
(a) means for receiving data characterizing the phenotype of the cell or cell population;
(b) means for analyzing the data to determine whether the cell or cell population possesses the following features:
(i) chromosomes that approach metaphase but fail to separate and maintain alignment compared to a control cell or cell population;
(ii) a bipolar spindle that is at least about 10% longer than a corresponding metaphase mitotic spindle from the control cell or cell population; and
(iii) during interphase the cell or population of cells exhibits a phenotype that is substantially similar to that of an interphase control cell or the interphase cells of a control cell population; and
(c) means for characterizing the cell or cell population as having a rice phenotype when the cell or cell population is found to possess at least the features specified in (b).
